SEQUENCE LISTING (9 sequences)

SEQ ID NO: 1 - protein, 244 aa, Artificial Sequence
  Variants of human adiponectin
  MISC_FEATURE (109)..(109): X at 109 can be either amino acid D, E, H, K, N, Q, R, or Y
  MISC_FEATURE (110)..(110): X at 110 can be either amino acid D, E, H, K, N, Q, R, S, or V
  MISC_FEATURE (111)..(111): X at 111 can be either amino acid D, E, K, N, Q, R, Y, or H
  MISC_FEATURE (122)..(122): X at 122 can be either amino acid D, E, H, N, R, S, or Y
  MISC_FEATURE (125)..(125): X at 125 can be either amino acid D, E, H, K, N, Q, R, S, T, or I
  MISC_FEATURE (128)..(128): X at 128 can be either amino acid A, D, E, H, K, N, Q, R, S, T, or M
  MISC_FEATURE (135)..(135): X at 135 can be either amino acid D, E, H, K, N, Q, R, or I
  MISC_FEATURE (152)..(152): X at 152 can be either amino acid A, N, S, or C
  MISC_FEATURE (182)..(182): X at 182 can be either amino acid A, D, E, K, N, Q, R, S, T, or M
  MISC_FEATURE (184)..(184): X at 184 can be either amino acid D, H, K, N, R, or F
  MISC_FEATURE (207)..(207): X at 207 can be either amino acid D, E, H, K, N, Q, R, S, or V
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAXXXRSAFSVGLETXVTXPNXPIRFTKXFYNQQNHYDGSTGKFHXNIPGLYYFAYHITVYMKDVKVSLFKKDKAXLXTYDQYQENNVDQASGSVLLHLEXGDQVWLQVYGEGERNGLYADNDNDSTFTGFLLYHDTN

SEQ ID NO: 2 - protein, 66 aa, Artificial Sequence
  Synthetic peptide
GPPGPPGPPGPPGPPGPPGPPGPPGPPGPPGPPGPPGPPGPPGPPGPPGPPGPPGPPGPPGPPGPP

SEQ ID NO: 3 - protein, 247 aa, Mouse
MLLLQALLFLLILPSHAEDDVTTTEELAPALVPPPKGTCAGWMAGIPGHSGHNGTPGRDGRDGTPGEKGEKGDAGLLGPKGETGDVGMTGAEGPRGFPGTPGRKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLFHDTN

SEQ ID NO: 4 - protein, 244 aa, Rat
MLLLQALLFLLILPSHEGITATEGPGALVPPPKETCAGWMAGIPGYPGHNGIPGRDGRDGTPGEKGEKGDAGVLGPKGDPGDAGMTGAEGPRGFPGTPGRKGEPGEAAYMYHSAFSVGLETRVTVPNVPIRFTKIFYNQQNHYDGSTGKFHCNIPGLYYFSYHITVYMKDVKVSLFKKDKAVLFTYDQYQEKNVDQASGSMLLHLEVGDQVWLQVYGEGDNNGLYADNVNDSTFTGFLLYHDTN

SEQ ID NO: 5 - protein, 243 aa, Macaque
MLLGAVLLLLALPSHGQDTTTQGPGVLLPLPKGACTGWMAGIPGHPGHNGVPGRDGRDGTPGEKGEKGDPGLIGPKGDTGETGVTGAEGPRGFPGIQGRKGEPGEGAYVYRSAFSVGLETYVTVPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 6 - protein, 244 aa, Dog
MLLLRAVLLLLVLPAHGQDSVAEGPGVLLPLPKGACPGWMAGIPGHPGHNGTPGRDGRDGTPGEKGEKGDAGLVGPKGDTGETGVTGVEGPRGFPGTPCRKGEPGESAYVHRSAFSVGLESRITVPNVPIRFTKIFYNLQNHYDGTTGKFHCNIPGLYYFSYHITVYLKDVKVSLYKKDKAMLFTYDQYQEKNVDQASGSVLLHLEVGDQVWLQVYGDGDSYGIYADNVNDSTFTGFLLYHDTN

SEQ ID NO: 7 - protein, 243 aa, Boar
MLLLGAVLLLLALPSLGQETTEKPGALLPMPKGACAGWMAGIPGHPGHNGTPGRDGRDGVPGEKGEKGDTGLTGPKGDTGESGVTGVEGPRGFPGIPGRKGEPGESAYVYRSAFSVGLETRVTVPNMPIRFTKIFYNQQNHYDVTTGKFHCNIPGLYYFSFHITVYLKDVKVSLYKKDKAVLFTYDQYQDKNVDQASGSVLLYLEKGDQVWLQAYGDEENNGVYADNVNDSIFTGFLLYHNIE

SEQ ID NO: 8 - protein, 240 aa, Cow
MLLQGALLLLLALPSHGEDNMEDPPLPKGACAGWMAGIPGHPGHNGTPGRDGRDGTPGEKGEKGDAGLLGPKGETGDVGMTGAEGPRGFPGTPGRKGEPGEAAYVYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYEGENHNGVYADNVNDSTFTGFLLYHNIVE

SEQ ID NO: 9 - protein, 244 aa, Chicken
MRGSVGFLLCSLLLALSGTEMADQADQSDPKMSCANWMGGAPGHPGHNGLPGRDGKDGKDGQKGDKGEPGLQGVKGDTGEKGATGAEGPRGFPGHMGMKGQKGESSYVYRSAFSVGLTERAPHPNVPIRFTKIFYNEQNHYDSSTGKFLCSIPGTYFFAYHLTVYMTDVKVSLYKKDKAVIFTYDQFQENNVDQASGSVLLHLSLGDEVWLQVYGEGNNNGVYADNINDSTFMGFLLYPDTDDR

(19) United States
(12) Patent Application Publication
(10) Pub. No.: US 2007/0015909 A1
(43) Pub. Date: Jan. 18, 2007

(54) Adiponectin variants

(75) Inventors: Darian Cash, Los Angeles, CA (US); Arthur J. Chirino, Camarillo, CA (US); John R. Desjarlais, Pasadena, CA (US); Sergei A. Ezhevsky, San Diego, CA (US); Gregory L. Moore, Pasadena, CA (US); Duc-Hahn Thi Nguyen, Sylmar, CA (US); Jonathan Zalevsky, Riverside, CA (US)

Correspondence Address: HOGAN & HARTSON L.L.P., 1999 AVENUE OF THE STARS, SUITE 1400, LOS ANGELES, CA, 90067, US

(73) Assignee: XENCOR

(21) Appl. No.: 11/328,901

(22) Filed: Jan. 9, 2006

Related U.S. Application Data
(60) Provisional application No. 60/642,476, filed on Jan. 7, 2005.
(60) Provisional application No. 60/650,411, filed on Feb. 3, 2005.
(60) Provisional application No. 60/698,358, filed on Jul. 11, 2005.
(60) Provisional application No. 60/720,768, filed on Sep. 26, 2005.
(60) Provisional application No. 60/733,137, filed on Nov. 2, 2005.

(51) Int. Cl. C07K 14/715; A61K 38/17; C07H 21/04

(52) U.S. Cl. 530/350; 536/23.5; 514/12

(57) ABSTRACT

Adiponectin variants comprising one or more amino acid modifications to corresponding wild-type adiponectins at positions having predetermined hydrophobicity, predetermined polarity, predetermined electrostatic potential, Met, aromatic amino acid, Cys corresponding to position 152 of SEQ ID NO:1, amino acid affecting isoelectric point of the wild-type or variant adiponectin, amino acid affecting beta sheet formation, helix capping, or dipole interactions, or a combination thereof, wherein the adiponectin variants exhibit improved stability, solubility or soluble expression, expression yield, the ability to induce phosphorylation of AMPK, or a combination thereof, as compared to the corresponding wild-type adiponectins.

### CROSS-REFERENCE TO PRIOR APPLICATIONS

This application claims the benefit of prior U.S. Provisional Application No. 60/642,476 filed Jan. 7, 2005, U.S. Provisional Application No. 60/650,411 filed Feb. 3, 2005, U.S. Provisional Application No. 60/698,358 filed Jul. 11, 2005, U.S. Provisional Application No. 60/720,768 filed Sep. 26, 2005, and U.S. Provisional Application No. 60/733,137 filed Nov. 2, 2005, the contents of which are hereby incorporated by reference in their entirety.

### FIELD OF THE INVENTION

The present invention relates in general to adiponectin. More specifically, the invention relates to variants of human adiponectin and other C1q/TNF-α Related Proteins with improved properties, including increased recombinant protein expression levels, enhanced solubility or soluble expression and stability, lower immunogenicity, and improved pharmacokinetics and/or pharmacodynamics, as well as methods of making such variants and using them to treat diseases.

### BACKGROUND OF THE INVENTION

In addition to storing fat deposits, adipocytes secrete several cytokines important in regulating lipid and glucose metabolism in mammals. These so called “adipokines” include adiponectin (“Ad”), adipsin, leptin, and vaspin. In the literature, adiponectin has also been called GBP28, ApM1, ACRP30, AdipoQ, and OBG3. Unlike other adipokines, however, adiponectin serum levels are inversely correlated with obesity, insulin resistance and ischemic heart disease (Goldstein and Scalia (2004) The Journal of Clinical Endocrinology and Metabolism 89:2563-8, entirely incorporated by reference). While serum levels of adiponectin in normal humans typically range from 2 to 10 ug/ml, levels of circulating Ad are dramatically reduced in obese or diabetic individuals. Accordingly, Ad replacement therapy has been suggested as a possible treatment to reverse insulin resistance in type II diabetics and to ameliorate vascular atherosclerosis in at-risk cardiac patients.

Ad treatment has been shown to mobilize glucose and fatty acid clearance as well as to induce insulin sensitivity in both normal and insulin resistant tissues (Wu et al. (2003) Diabetes 52:1355-63; Fruebis et al. (2001) PNAS 98:2005-10; Berg et al. (2002) TRENDS in Endocrinology and Metabolism 13:84-9; all entirely incorporated by reference). These effects appear to be due to Ad-induced activation of transport proteins and metabolic enzymes in both the skeletal muscle and liver. Ad is known to stimulate the phosphorylation and subsequent activation of 5′-AMP-activated protein kinase (AMPK), acetyl coenzyme A carboxylase (ACC) (Yamauchi et al. (2002) Nature Medicine 8:1288-95, entirely incorporated by reference), and also activate the pPAR family of steroid hormone receptors (Yamauchi et al. (200) Journal of Biological Chemistry 278:2461-8, entirely incorporated by reference). Additional studies have shown that Ad has both cardioprotective and anti-inflammatory properties (Shimada et al (2004) Clinica. Chemica. Acta. 344:1-12; Hug and Lodish (2005) Current Opinion in Pharmacology 5:129-34, all entirely incorporated by reference). Recent studies show that adiponectin can interact with and alter the activity of several growth factors including platelet derived growth factor BB (PDGF-BB), heparin-binding epidermal growth factor-like growth factor (HB-EGF), and basic fibroblast growth factor (basic FGF) (Wang et al. (2005) Journal of Biological Chemistry 280:18341-7, entirely incorporated by reference).

Ad is a 30 kD glycoprotein consisting of an N-terminal collagen-like domain containing multiple G-X-X-G repeats and a C-terminal domain structurally resembling the globular portions of the C1Q and TNF superfamily members. At least two proteolytic cleavage sites are located between the collagen and C1Q-like domains. Both full length and proteolytically cleaved forms are found in human serum. Globular portions of Ad (“globular” Ad or gAd) form trimeric structures, while full length Ad (Ad) is capable of forming trimers, hexamers, and additional higher order oligomers. Mutation of the cysteine residue located in the collagen domain (conserved in all known mammalian Ad) abolishes hexamer and high-order oligomer formation.

Homologous proteins to Ad include, but are not limited to, mouse C1q/TNF-α Related Proteins 1 (CTRP1), CTRP2, CTRP3, CTRP4, CTRP5, CTRP6 and CTRP7. At least one of these proteins (CTRP2) is able to stimulate fatty acid oxidation in skeletal muscle, thus resembling the functional properties of Ad (Wong et al. (2004) Proc. Natl. Acad. Sci. 101:10302-7, entirely incorporated by reference).

Several Ad polymorphisms have been discovered within particular human populations. The severity of the phenotype depends on the position of the mutation. For example, the G84R, G90S, Y111H, and I164T mutations cause diabetes and hypoadiponectinemia as a result of a failure to form higher order oligomers that are likely important in regulating insulin sensitivity by the liver (Waki et al. (2003) J. Biol. Chem. 278:40352-63, entirely incorporated by reference). Functionally benign polymorphisms include R221S and H241P.

Based on their amino acid sequences, both known Ad receptors (AdipoR1 and AdipoR2) are predicted to contain seven transmembrane alpha helices but are not related to G-coupled protein receptors (Yamauchi et al. (2003) Nature 423:762-9, entirely incorporated by reference). Although AdipoR1 and AdipoR2 are homologous (>67% identity), their relative affinities to Ad and gAd differ. AdipoR1, expressed predominantly in skeletal muscle, binds to gAd with higher affinity than Ad, while AdipoR2, expressed predominantly in liver, binds preferentially to Ad. In vivo results in mice suggest that trimeric gAd may be more effective at reducing weight and improving insulin sensitivity than hexameric and higher order oligomeric forms of Ad (Yamauchi et al. (2001) Nature Medicine 7:941-6, entirely incorporated by reference).

### SUMMARY OF THE INVENTION

The present invention provides novel adiponectin variants that are optimized for increased levels of recombinant protein expression, improved solubility or soluble expression and stability, lower immunogenicity, and improved pharmacokinetics and/or pharmacodynamics.

Accordingly, the invention features an adiponectin variant comprising one or more amino acid modifications to a corresponding wild-type adiponectin at positions having predetermined hydrophobicity, predetermined polarity, predetermined electrostatic potential, Met, aromatic amino acid, Cys corresponding to position 152 of SEQ ID NO:1, amino acid affecting isoelectric point of the wild-type or variant adiponectin, amino acid affecting beta sheet formation, helix capping, or dipole interactions, or a combination thereof. The adiponectin variant exhibits improved stability, solubility or soluble expression, expression yield, the ability to induce phosphorylation of 5′-AMP-activated protein kinase (AMPK), or a combination thereof, as compared to the corresponding wild-type adiponectin.

In one aspect, the invention features a composition comprising a variant human adiponectin peptide. The variant comprises the formula of V(109)-V(110)-V(111)-F(112)-F(113-121)-V(122)-F(123-124)-V(125)-F(126-127)-V(128)-F(129-134)-V(135)-F(136-151)-V(152)-F(153-163)-F(164)-F(165-181)-V(182)-F(183)-V(184)-F(185-206)-V(207)-F(208-220)-F(221)-F(222-223)-V(224)-V(225)-F(226)-V(227)-F(228)-V(229). V(109) is selected from the group consisting of: the wild-type amino acid V; any of variant amino acids D, E, H, K, N, Q, and R; and, a deletion of V109; V(110) is selected from the group consisting of: the wild-type amino acid V; any of variant amino acids D, E, H, K, N, Q, R, and S; and, a deletion of V110; V(111) is selected from the group consisting of: the wild-type amino acids Y and H; any of variant amino acids D, E, N, R, and S; and, a deletion of Y122; F(112) is selected from the group consisting of the wild-type amino acids R and C; F(113-121) is selected from the group consisting of: the wild-type amino acid sequence SAFSVGLET; and, a deletion of any of S113, A114, F115, S116, V117, G118, L119, E120, and T121; V(122) is selected from the group consisting of: the wild-type amino acid Y; any of variant amino acids D, E, H, N, R, and S; and, a deletion of Y122; F(123-124) is selected from the group consisting of: the wild-type amino acid sequence VT; and, a deletion of any of V123 and T124; V(125) is selected from the group consisting of: the wild-type amino acid I; any of variant amino acids D, E, H, K N, Q, R, S, and T; and, a deletion of I125; F(126-127) comprises the wild-type amino acid sequence PN; V(128) is selected from the group consisting of: the wild-type amino acid M; and any of variant amino acids A, D, E, H, K, N, Q, R, S, and T; F(129-134) comprises the wild-type amino acid sequence PIRFTK; V(135) is selected from the group consisting of: the wild-type amino acid I; and, any of variant amino acids D, E, H, K, N, Q and R; F(136-151) comprises the wild-type amino acid sequence FYNQQNHYDGSTGKFH; V(152) is selected from the group consisting of: the wild-type amino acid C; and, any of variant amino acids A, N and S; F(153-163) comprises the wild-type amino acid sequence NIPGLYYFAYH; F(164) is selected from the group consisting of the wild-type amino acid I and T; F(165-181) comprises the wild-type amino acid sequence TVYMKDVKVSLFKKDKA; V(182) is selected from the group consisting of: the wild-type amino acid M; and, any of variant amino acids A, D, E, K, N, Q, R, S, and T; F(183) comprises the wild-type amino acid L; V(184) is selected from the group consisting of: the wild-type amino acid F; and, any of variant amino acids D, H, K, N and R; F(185-206) comprises the wild-type amino acid sequence TYDQYQENNVDQASGSVLLHLE; V(207) is selected from the group consisting of: the wild-type amino acid V; and, any of variant amino acids D, E, H, K, N, Q, R, and S; F(208-220) comprises the wild-type amino acid sequence GDQVWLQVYGEGE; F(221) is selected from the group consisting of the wild-type amino acids R and S; F(222-223) comprises the wild-type amino acid sequence NG; V(224) is selected from the group consisting of: the wild-type amino acid L; and, any of variant amino acids D, E, H, K, N, Q, R and S; V(225) is selected from the group consisting of: the wild-type amino acid Y; and, any of variant amino acids D, E, H, K, N, Q, R and S; F(226) comprises the wild-type amino acid A; V(227) is selected from the group consisting of: the wild-type amino acid D; and, any of variant amino acids H, K and R; F(228) comprises the wild-type amino acid N; or V(229) is selected from the group consisting of: the wild-type amino acid D; and, any of variant amino acids H, K and R.

In one embodiment, the variant contains a substitution selected from the group consisting of 122H; 122S; 125E; 125H; 125T; 184H; 207E; and 207K.

In another embodiment, the variant comprises at least two modifications such as substitutions.

In some embodiments, the solubility or soluble expression of the variant is improved by at least n-fold, where n is any number between 2 and 2000. For example, the solubility or soluble expression of the variant may be improved by at least 30-, 100-, 300, and 1000-fold.

In some embodiments, the expression yield of the variant is improved by at least n-fold, where n is any number between 2 and 10000. For example, the expression yield of the variant may be improved by at least 2-, 5-, 10-, 50-, 100-, 300-, 500-, 1000-, 3000-, and 10000-fold.

In some embodiments, the ability of the variant to induce phosphorylation of AMPK in muscle cells is improved by at least 30% or 100%.

The corresponding wild-type adiponectin may be a human adiponectin (SEQ ID NO:1), and the variant may include one or more amino acid modifications at position 109, 110, 115, 122, 123, 125, 128, 130, 132, 135, 150, 152, 160, 164, 166, 171, 173, 175, 182, 184, 205, 207, 211, 213, 215, 224, 225, 227, 229, or 234 of SEQ ID NO:1. Alternatively, the corresponding wild-type adiponectin may be a non-human adiponectin.

In another aspect, the invention features a composition comprising a polynucleotide encoding the adiponectin variant described above.

Also within the invention is a composition comprising a variant adiponectin peptide, the solubility or soluble expression of which is improved by at least n-fold, where n is any number between 2 and 2000. For example, the solubility or soluble expression of the variant may be improved by at least 30-, 100-, 300, and 1000-fold.

Especially preferred modifications to Ad include, but are not limited to, the following substitutions: Y109D, Y109E, Y109H, Y109K, Y109N, Y109Q, Y109R, V110D, V110E, V110H, V110K, V110N, V110Q, V110R, V110S, Y111D, Y111E, Y111K, Y111N, Y111Q, Y111R, Y122D, Y122E, Y122H, Y122N, Y122R, Y122S, I125D, I125E, I125H, I125K, I125N, I125Q, I125R, I125S, M128A, M128D, M128E, M128H, M128K, M128N, M128Q, M128R, M128S, M128T, I135D, I135E, I135H, I135K, I135N, I135Q, I135R, C152A, C152N, C152S, M182A, M182D, M182E, M182K, M182N, M182Q, M182R, M182S, M182T, F184D, F184H, F184K, F184N, F184R, V207D, V207E, V207H, V207K, V207N, V207Q, V207R, V207S, L224D, L224E, L224H, L224K, L224N, L224Q, L224R, L224S, Y225D, Y225E, Y225H, Y225K, Y225N, Y225Q, Y225R, Y225S, D227H, D227K, D227R, D229H, D229K, D229R, or a combination thereof.

The above-mentioned and other features of this invention and the manner of obtaining and using them will become more apparent, and will be best understood, by reference to the following description, taken in conjunction with the accompanying drawings. These drawings depict only typical embodiments of the invention and do not therefore limit its scope.

### BRIEF DESCRIPTION OF THE FIGURES

FIG. 1 shows the full-length human adiponectin amino acid sequence (SEQ ID NO:1, Genbank accession No. Q15848, residues 1-244), the collagen region is underlined.

FIG. 2 shows the alignment of full-length human adiponectin (SEQ ID NO:1) and collagen sequences.

FIG. 3 shows ClustalW alignment of full-length human, mouse, rat, rhesus macaque, dog, boar, cow, and chicken adiponectin.

FIG. 4 is a graph that demonstrates the relationship between amino acid surface exposure and the relative hydrophobicity of that amino acid.

FIG. 5 shows SDS-PAGE analysis of 34 single amino acid substitution-containing gAd variants. Proteins were expressed in E. coli and lysates were prepared in the presence of detergent.

FIG. 6 shows solubility or soluble expression analyses of selected single amino acid substitution-containing gAd variants. Proteins were expressed in E. coli and lysates were prepared under detergent-free conditions.

FIG. 7 shows SDS-PAGE analysis of eight single amino acid and 23 double amino acid substitution-containing gAd variants. Proteins were expressed in E. coli and lysates were prepared in the presence of detergent.

FIG. 8 shows solubility or soluble expression analyses of selected single and double amino acid substitution-containing gAd variants. Proteins were expressed in E. coli and lysates were prepared under detergent-free conditions.

FIG. 9 shows an SDS-PAGE that contained the detergent-free soluble lysates from native and V207E/I125E gAd.

FIG. 10 shows phase contrast time-course images of mouse C2C12 myotube differentiation.

FIG. 11 shows treatment of C2C12 myotubes with gAd variants and controls.

FIG. 12 shows that treatment of differentiated human muscle cells with gAd variants induces AMPK phosphorylation.

FIG. 13 shows three-dimensional structure of low energy core design of globular adiponectin domain (2^{nd} lowest energy sequence solution in Table 19). Dark grey balls-and-sticks depict wild type side-chains (I164 and V166) in their native conformations while light grey atoms depict low-energy amino acid substitutions I164V and V166F.

FIG. 14 shows optimization of PolyEthylene Glycol (PEG) sites for adiponectin using a PEG of molecular weight of 2000 and using a cysteine-maleimide attachment moiety. Potential attachment sites were evaluated using a population of 500 self-avoiding PEG chains. The percentage of chains that did not clash with the gAd structure are plotted for each position in gAd. The percentage of non-clashing chains was plotted for both the monomer (top chart) and trimer gAd structures.

### DETAILED DESCRIPTION OF THE INVENTION

In order that the invention may be more completely understood, several definitions are set forth below. Such definitions are meant to encompass grammatical equivalents.

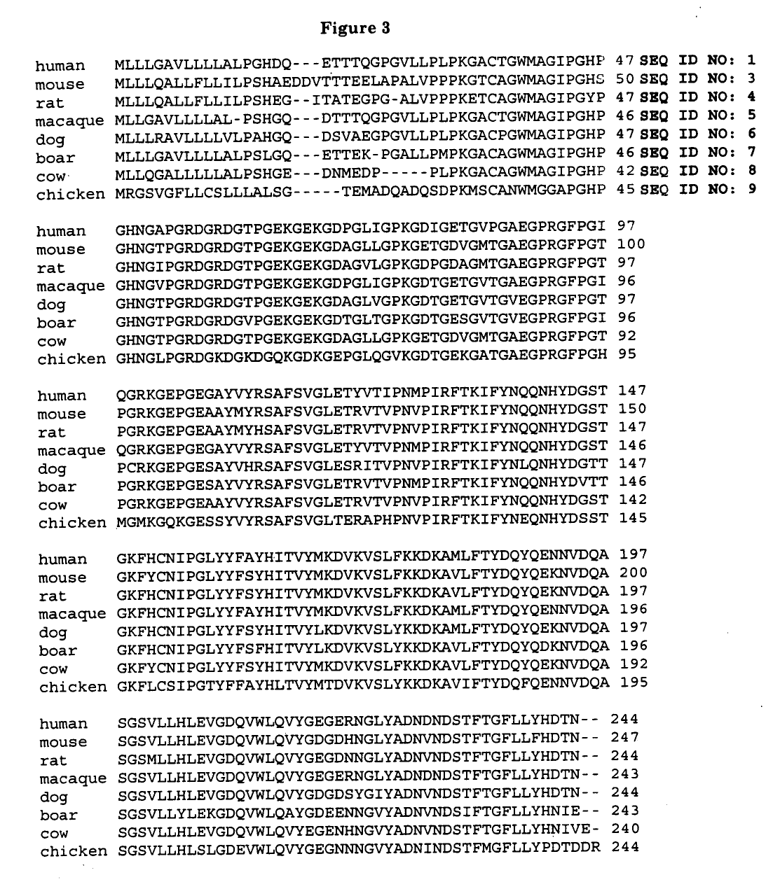

By “adiponectin” herein is meant a polypeptide that is primarily derived in adipocytes and is an ortholog of any sequence shown in FIG. 3, including fragments of naturally-occurring adiponectin, especially fragments containing the globular domain of adiponectin.

By “adiponectin variant” herein is meant a polypeptide that is functionally equivalent to adiponectin but contains modifications to a naturally-occurring adiponectin sequence.

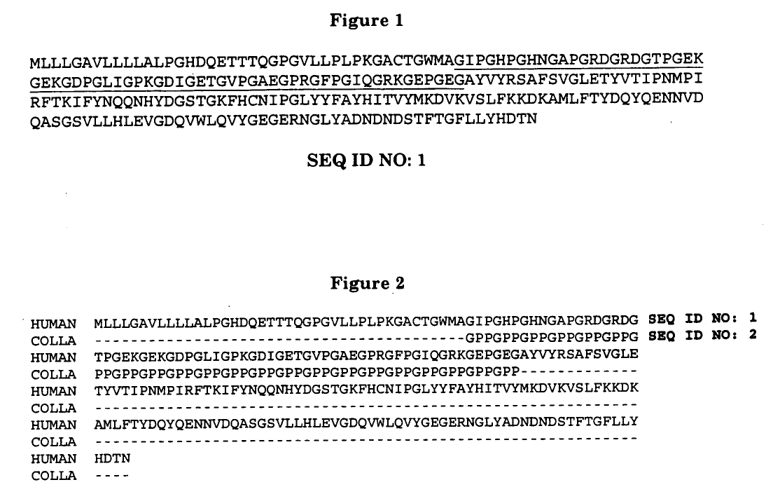

By “globular domain” herein is meant, in the context of Ad, the C1q/TNF-α-like domain and not including the collagen domain. This region can include but is not limited to residues 108-244 of the human Ad precursor form (SEQ ID NO:1, FIG. 1).

By “hydrophobic residues” and grammatical equivalents are meant valine, isoleucine, leucine, methionine, phenylalanine, tyrosine, tryptophan, and functional equivalents thereof.

By “polar residues” and grammatical equivalents herein are meant aspartic acid, asparagine, glutamic acid, glutamine, lysine, arginine, histidine, serine, and functional equivalents thereof.

By “protein properties” herein are meant physical, chemical, and biological properties including but not limited to physical properties (including molecular weight, hydrodynamic properties such as radius of gyration, net charge, isoelectric point, and spectral properties such as extinction coefficient), structural properties (including secondary, tertiary, and quaternary structural elements), stability (including thermal stability, stability as a function of pH or solution conditions, storage stability, and resistance or susceptibility to ubiquitination, proteolytic degradation, or chemical modifications such as methionine oxidation, asparagine and glutamine deamidation, sidechain racemerization or epimerization, and hydrolysis of peptide bonds), solubility (including susceptibility to aggregation under various conditions, oligomerization state, and crystallizability), kinetic and dynamic properties (including flexibility, rigidity, folding rate, folding mechanism, allostery, and the ability to undergo conformational changes and correlated motions), binding affinity and specificity (to one or more molecules including proteins, nucleic acids, polysaccharides, lipids, and small molecules, and including affinities and association and dissociation rates), enzymatic activity (including substrate specificity; association, reaction, and dissociation rates; reaction mechanism; and pH profile), ammenability to synthetic modification (including PEGylation and attachment to other molecules or surfaces), expression properties (such as yield in one or more expression hosts, soluble versus inclusion body expression, subcellular localization, ability to be secreted, and ability to be displayed on the surface of a cell), processing and posttranslational modifications (including proteolytic processing, N- or C-linked glycosylation, lipidation, sulfation, and phosphorylation), pharmacokinetic and pharmacodynamic properties (including bioavailability following subcutaneous, intramuscular, oral, or pulmonary delivery; serum half-life, distribution, and mechanism and rate of elimination), and ability to induce altered phenotype or changed physiology (including immunogenicity, toxicity, ability to signal or inhibit signaling, ability to stimulate or inhibit cell proliferation, differentiation, or migration, ability to induce apoptosis, and ability to treat disease).

By “solubility” and grammatical equivalents herein is meant the maximum possible concentration of protein, in the desired or physiologically appropriate oligomerization state, in a solution of specified condition (i.e., pH, temperature, concentration of any buffer components, salts, detergents, osmolytes, etc.).

By “improved solubility” and grammatical equivalents herein is meant an increase in the maximum possible concentration of protein, in the desired or physiologically appropriate oligomerization state, in solution. For example, if the naturally occurring protein can be concentrated to 1 mM and the variant can be concentrated to 5 mM under the same solution conditions, the variant can be said to have improved solubility. In a preferred embodiment, solubility is increased by at least a factor of 2, with increases of at least 5-fold or 10-fold being especially preferred. As will be appreciated by those skilled in the art, solubility is a function of solution conditions. For the purposes of this invention, solubility should be assessed under solution conditions that are pharmaceutically acceptable. Specifically, pH should be between 6.0 and 8.0, salt concentration should be between 50 and 250 mM. Additional buffer components such as excipients may also be included; although it is preferred that albumin is not required.

By “soluble expression” and grammatical equivalents herein is meant the amount of target protein in a crude supernatant prepared in the absence of detergent. For example, a target protein is expressed in an appropriate expression system, cells harvested and lysed in the absence of detergent, and a crude supernatant is prepared by standard methods. The amount of target protein in the crude supernatant is the soluble expressed protein.

By “improved soluble expression” and grammatical equivalents herein is meant an increase in the quantity of variant protein in a crude supernatant prepared in the absence of detergent relative to a parent protein.

By “modification” and grammatical equivalents is meant one or more insertions, deletions, or substitutions to a protein or nucleic acid sequence. The insertions and substitutions include naturally- or non-naturally-occurring amino acids and nucleotides, as well as their functional equivalents.

By “naturally occurring” or “wild type” or “wt” and grammatical equivalents thereof herein is meant an amino acid sequence or a nucleotide sequence that is found in nature, including allelic variations. In a preferred embodiment, the wild type sequence is the most prevalent human sequence. However, the wild type Ad nucleic acids and proteins may be a less prevalent human allele or Ad nucleic acids and proteins from any number of organisms, including but not limited to rodents (rats, mice, hamsters, guinea pigs, etc.), primates, and farm animals (including sheep, goats, pigs, cows, horses, etc).

By “expression yield” and grammatical equivalents herein is meant the amount of protein, preferably in mg/L or PCD (picograms per cell per day) that is produced or secreted under a given expression protocol (that is, a specific expression host, transfection method, media, time, etc.).

By “improved expression yield” and grammatical equivalents herein is meant an increase in expression yield, relative to a wild type or parent protein, under a given set of expression conditions. In a preferred embodiment, at least a 50% improvement is achieved, with improvements of at least 100%, 5-fold, 10-fold, or more being especially preferred.

As mentioned previously, serum levels of endogenous Ad in healthy individuals typically lies between 2 to 10 ug/ml, a rather large amount relative to other serum proteins. If these amounts are required for efficacious replacement therapy to treat, for example, obesity or diabetes, large quantities of highly soluble, non-aggregation-prone protein will be required. This will aid Ad administration to patients and will likely lead to efficient product manufacturing.

The invention is based, at least in part, upon the unexpected discovery that adiponectin can be modified such that the physical properties and/or biological activities of the polypeptide are improved. Accordingly, the invention provides an adiponectin variant with improved physical properties (e.g., stability, solubility or soluble expression, and expression yield) and/or biological activities (e.g., the ability to induce phosphorylation of AMPK), as compared to the corresponding wild-type adiponectin. The variant comprises one or more amino acid modifications to the corresponding wild-type adiponectin. The modifications can be made at the following positions:

(1) Positions that have predetermined hydrophobicity and percent exposure. Hydrophobicity and percent exposure of an amino acid can be determined as described below or by any method well known in the art. In preferred embodiments, the top 10% of exposed hydrophobic amino acids are selected for modification.

(2) Positions that have predetermined polarity. Examples of polar residues include aspartic acid, asparagine, glutamic acid, glutamine, lysine, arginine, histidine, and serine. In some embodiments, charged polar residues are substituted for neutral polar residues occurring naturally in adiponectin.

(3) Positions that have predetermined electrostatic potential. Electrostatic potential of an amino acid can be determined as described below or by any method well known in the art. In preferred embodiments, amino acids with electrostatic potentials greater than 0.5 kcal/mol or less than −0.5 kcal/mol are selected for modification.

(4) Positions that have Met, e.g., positions 40, 128, 168, and 182 of SEQ ID NO:1.

(5) Positions that have hydroxyPro, e.g., positions 44, 47, 53, 62, 71, 86, 95, and 104 of SEQ ID NO:1.

(6) Positions that have an aromatic amino acid, e.g., positions 46, 49, and 94 of SEQ ID NO:1.

(7) Cys corresponding to position 152 of SEQ ID NO:1.

(8) Positions that have PEGylation site, e.g., positions 108, 109, 110, 120, 127, 133, 136, 137, 139, 141, 146, 170, 179, 180, 184, 186, 188, 189, 191, 192, 196, 202, 204, 206, 207, 208, 218, 220, 221, 223, 224, 225, 226, 227, 229, 240, 243, and 244 of SEQ ID NO:1.

(9) Positions that have amino acids affecting isoelectric point of the wild-type or variant adiponectin. Such amino acids can be determined by any method well known in the art. Examples of such amino acids include aspartic acid, glutamic acid, histidine, lysine, arginine, tyrosine, and cysteine.

(10) Positions that have amino acids affecting beta sheet formation, helix capping, or dipole interactions. Such amino acids can be determined by any method well known in the art.

Strategies for Improving Solubility or Soluble Expression

A variety of strategies may be utilized to design adiponectin variants with improved solubility or soluble expression and expression yield. In a preferred embodiment, one or more of the following strategies are used: 1) reduce hydrophobicity by substituting one or more solvent-exposed hydrophobic residues with suitable polar residues, 2) increase polar character by substituting one or more neutral polar residues with charged polar residues, 3) increase protein stability, for example by one or more modifications that improve packing in the hydrophobic core, increase beta sheet forming propensity, improve helix capping and dipole interactions, or remove unfavorable electrostatic interactions (increasing the stability of a protein may improve solubility or soluble expression by decreasing the population of partially folded or misfolded states that are prone to aggregation), 4) modify one or more residues that can affect the isoelectric point of the protein (that is, aspartic acid, glutamic acid, histidine, lysine, arginine, tyrosine, and cysteine residues). Protein solubility or soluble expression is typically at a minimum when the isoelectric point of the protein is equal to the pH of the surrounding solution. Modifications that perturb the isoelectric point of the protein away from the pH of a relevant environment, such as serum, may therefore serve to improve solubility or soluble expression. Furthermore, modifications that decrease the isoelectric point of a protein may improve injection site absorption (Holash et. al. (2002) Proc. Nat. Acad. Sci. USA 99:11393-8, entirely incorporated by reference), 5) truncation of N- or C-terminal residues, 6) addition or chemical attachment of solubility or soluble expression tags (e.g., peptide or chemical moieties that have high solubility or soluble expression), 7) PEGylation, and 8) introduction of glycosylation sites. Additional strategies may involve the use of directed evolution methods to discover variants that improve solubility or soluble expression (see, for example, Waldo (2002) Curr Opin Chem Biol. 7(1):33-8).

Strategies for Improving Expression Yield

A number of nucleic acid properties and protein properties may influence expression yields; furthermore, the expression host and expression protocol contribute to yields. Any of these parameters may be optimized to improve expression yields. Also, expression yield may be improved by the incorporation of one or more mutations that confer improved stability and/or solubility or soluble expression, as discussed further below. Furthermore, interactions between the pro-domain and the mature domain may influence folding efficiency, and so the pro-domain may also be targeted for modification.

In an alternate embodiment, if expression is in a eukaryotic system, nucleic acid properties are optimized to improve expression yields using one or more of the following strategies: 1) replace imperfect Kozak sequence, 2) reduce 5′ GC content and secondary structure of the RNA, 3) optimize codon usage, 4) use an alternate leader sequence, 5) include a chimeric intron, or 6) add an optimized poly-A tail to the C-terminus of the message. In another preferred embodiment, protein properties are optimized to improve expression yields using one or more of the following strategies: 1) optimize the signal sequence, 2) optimize the proteolytic processing site, 3) replace one or more cysteine residues in order to minimize formation of improper disulfide bonds, 4) improve the rate or efficiency of protein folding, or 5) increase protein stability, especially proteolytic stability. In an alternate preferred embodiment, alternate pro-domain sequences are used. For example, the pro-domain from adiponectin-2 may be used to aid in the expression of adiponectin-4 (Wozney et al. (1988) Science 242:1528-34, incorporated entirely by reference). Pro-domains that may be used include but are not limited to the pro-domains from any TNF-alpha superfamily sequence pro-domain. The pro-domain may be expressed in cis or in trans.

Strategies for Reducing Immunogenicity

Several methods have been developed to modulate the immunogenicity of proteins. In some cases, PEGylation has been observed to reduce the fraction of patients who raise neutralizing antibodies by sterically blocking access to antibody agretopes (see for example, Hershfield et al. (1991) PNAS 88:7185-9; Bailon et al. (2001) Bioconjug. Chem. 12:195-202; He et al. (1999) Life Sci. 65:355-68, all entirely incorporated by reference). Methods that improve the solution properties of a protein therapeutic may also reduce immunogenicity, as aggregates have been observed to be more immunogenic than soluble proteins. Additional methods for reducing immunogenicity include removal of potential MHC agretopes and/or T-cell epitopes, and modifications to decrease antigenicity.

Rational PEGylation

In another preferred embodiment, one or more cysteine, lysine, histidine, or other reactive amino acids are designed into variant Ad or gAd proteins in order to incorporate PEGylation sites. It is also possible to remove one or more cysteine, lysine, histidine, or other reactive amino acids in order to prevent the incorporation of PEGylation sites at specific locations. For example, in a preferred embodiment, non-labile PEGylation sites are selected to be well removed from the Ad trimerization interface and any required receptor binding sites in order to minimize loss of activity.

Protein Design and Engineering Methods

A number of methods can be used to identify modifications (that is, insertion, deletion, or substitution mutations) that will yield Ad variants with improved solubility or soluble expression and retained or improved ability to regulate cell proliferation, migration, differentiation, and apoptosis. These methods include, but are not limited to, sequence profiling (Bowie and Eisenberg (1991) Science 253:164-70), rotamer library selections (Dahiyat and Mayo (1996) Protein Sci 5:895-903; Dahiyat and Mayo (1997) Science 278:82-7; Desjarlais and Handel (1995) Prot. Sci. 4:2006-18; Harbury et al. (1995) Proc. Nat. Acad. Sci. USA 92:8408-12; Kono et al. (1994) Proteins 19:244-55; Hellinga and Richards (1994) Proc. Nat. Acad. Sci. USA 91:5803-7); and residue pair potentials (Jones (1994) Prot. Sci. 3:567-74), all entirely incorporated by reference.

In a preferred embodiment, one or more sequence alignments of Ads and related proteins is analyzed to identify residues that are likely to be compatible with each position. In a preferred embodiment, the PFAM, BLAST, or ClustalW alignment algorithms are used to generate alignments of the multi-species Ad orthologs, the C1q/TNF-a superfamily, or additional CTRP family members, homologs, orthologs or paralogs. For each variable position, suitable substitutions may be defined as those residues that are observed at the same position in homologous sequences. Especially preferred substitutions are those substitutions that are frequently observed in homologous sequences.

In an especially preferred embodiment, rational design of improved Ad variants is achieved by using Protein Design Automation® (PDA®) technology; see U.S. Pat. Nos. 6,188,965; 6,269,312; 6,403,312; 6,708,120; WO98/47089; U.S. Ser. No. 09/058,459; 09/127,926; 60/104,612; 60/158,700; 09/419,351; 60/181,630; 60/186,904; 09/782,004; 09/927,790; 60/347,772; 10/218,102; 60/345,805; 60/373,453; 60/374,035; and PCT/US01/218,102, all entirely incorporated by reference.

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure; and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions.

In a preferred embodiment, each polar residue is represented using a set of discrete low-energy side-chain conformations (see, for example, Dunbrack (2002) Curr. Opin. Struct. Biol. 12:431-40, entirely incorporated by reference). A preferred force field may include terms describing van der Waals interactions, hydrogen bonds, electrostatic interactions, and solvation, among others.

In a preferred embodiment, Dead-End Elimination (DEE) is used to identify the rotamer for each polar residue that has the most favorable energy (see Gordon et al. (2003) J. Comput Chem. 24:232-43, Goldstein (1994) Biophys. J. 66:1335-40, and Lasters and Desmet (1993) Prot. Eng. 6:717-22, all entirely incorporated by reference).

In an alternate embodiment, Monte Carlo can be used in conjunction with DEE to identify groups of polar residues that have favorable energies.

In a preferred embodiment, after performing one or more PDA® technology calculations, a library of variant proteins is designed, experimentally constructed, and screened for desired properties.

In an alternate preferred embodiment, a sequence prediction algorithm (SPA) is used to design proteins that are compatible with a known protein backbone structure (Raha et al. (2000) Protein Sci. 9:1106-19 and U.S. Ser. Nos. 09/877,695 and 10/071,859, all entirely incorporated by reference).

Library Selection

After performing one or more of the above-described calculations, a library comprising one or more preferred modifications may be proposed. The resulting library may be experimentally made and screened to confirm that one or more variants possess desired properties. In a preferred embodiment, the library comprises preferred point mutations identified using at least one of the above-described calculations.

In an alternate embodiment, the library is a combinatorial library, meaning that the library comprises all possible combinations of preferred residues at each of the variable positions. For example, if positions 3 and 9 are allowed to vary, preferred choices at position 3 are A, V, and I, and preferred choices at position 9 are E and Q, the library includes the following six variant sequences: 3A/9E, 3A/9Q, 3V/9E, 3V/9Q, 3I/9E, and 3I/9Q.

In an alternate embodiment, library construction is conducted in a master gAd sequence. The N-terminal truncation point may be at positions including but not limited to 100, 101, 102, 103, 104, 105, 106, 107, 108, 109, 110, 111, 112, 113, 114, 115, 116, 117, 118, 119, 120, 121, 122, 123, 124, 125 and 126.

Identifying Suitable Polar Residues for each Exposed Hydrophobic Position

In a preferred embodiment, solvent exposed hydrophobic residues are replaced with structurally and functionally compatible polar residues. Alanine and glycine may also serve as suitable replacements, constituting a reduction in hydrophobicity. Furthermore, mutations that increase polar character, such as Phe to Tyr, and mutations that reduce hydrophobicity, such as Ile to Val, may be appropriate.

In a preferred embodiment, solvent exposed hydrophobic residues in Ad are identified by analysis of a three-dimensional structure or model of Ad. In a preferred embodiment, solvent-accessible surface area is calculated using any of a variety of methods known in the art. In a preferred embodiment, solvent accessible surface area is combined with a hydrophobicity index. In a preferred embodiment, a hydrophobicity exposure index (HEI) for each residue is calculated by multiplying the residue's fractional solvent-exposure by the Fauchere and Pliska hydrophobicity index for that amino acid residue type (Fauchere and Pliska (1983) Eur. J. Med. Chem. 18:369-75, entirely incorporated by reference). In a preferred embodiment, residues with a positive HEI are selected for modification.

In a preferred embodiment, positions and variants for modification are selected according to the above criteria, and preferred variants produced experimentally then selected empirically, according to improved expression levels.

In a preferred embodiment, preferred suitable polar residues are defined as those polar residues: 1) whose energy in the optimal rotameric configuration, as determined using PDA® technology, is more favorable than the energy of the exposed hydrophobic residue at that position and 2) whose energy in the optimal rotameric configuration is among the most favorable of the set of energies of all polar residues at that position.

In a preferred embodiment, the polar residues that are included in the library at each variable position are deemed suitable by both PDA® technology calculations and by sequence alignment data. Alternatively, one or more of the polar residues that are included in the library are deemed suitable by either PDA® technology calculations or sequence alignment data.

Especially preferred modifications to Ad include, but are not limited to, the following substitutions: Y109D, Y109E, Y109H, Y109K, Y109N, Y109Q, Y109R, V110D, V110E, V110H, V110K, V110N, V110Q, V110R, V110S, Y111D, Y111E, Y111K, Y111N, Y111Q, Y111R, Y122D, Y122E, Y122H, Y122N, Y122R, Y122S, I125D, I125E, I125H, I125K, I125N, I125Q, I125R, I125S, M128A, M128D, M128E, M128H, M128K, M128N, M128Q, M128R, M128S, M128T, I135D, I135E, I135H, I135K, I135N, I135Q, I135R, C152A, C152N, C152S, M182A, M182D, M182E, M182K, M182N, M182Q, M182R, M182S, M182T, F184D, F184H, F184K, F184N, F184R, V207D, V207E, V207H, V207K, V207N, V207Q, V207R, V207S, L224D, L224E, L224H, L224K, L224N, L224Q, L224R, L224S, Y225D, Y225E, Y225H, Y225K, Y225N, Y225Q, Y225R, Y225S, D227H, D227K, D227R, D229H, D229K, D229R, or a combination thereof.

One skilled in the art will recognize that the above substitutions can be applied to optimize both full length and fragments of Ad as well as used to modify non-human adiponectin orthologs.

The invention also provides polynucleotides (DNA or RNA) comprising sequences encoding the adiponectin variants described above.

The adiponectin variants and polynucleotides of the invention can be made as described below or by any chemical synthesis or genetic engineering method well known in the art. The polynucleotides of the invention can be used to produce the adiponectin variants of the invention, which in turn can be used to generate antibodies.

The adiponectin variants and polynucleotides of the invention can be incorporated into pharmaceutical compositions. Such compositions typically include the adiponectin variants or polynucleotides and pharmaceutically acceptable carriers. A pharmaceutical composition is formulated to be compatible with its intended route of administration. See, e.g., U.S. Pat. No. 6,756,196, entirely incorporated by reference. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents, antibacterial agents such as benzyl alcohol or methyl parabens, antioxidants such as ascorbic acid or sodium bisulfite, chelating agents such as ethylenediaminetetraacetic acid, buffers such as acetates, citrates, or phosphates, and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In one embodiment, the adiponectin variants and polynucleotides of the invention are prepared with carriers that will protect the adiponectin variants and polynucleotides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, entirely incorporated by reference.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of active compound calculated to produce the desired therapeutic effect in association with the required pharmaceutical carrier.

Pharmaceutical compositions can be included in a container, pack, or dispenser together with instructions for administration to form packaged products. For example, a packaged product may comprise a container, an effective amount of a adiponectin variant or polynucleotide of the invention, and an insert associated with the container, indicating administering the compound for treating adiponectin-associated conditions.

Methods of Treatment

The invention additionally provides methods for treating adiponectin-associated conditions by administering to a subject in need thereof an effective amount of a composition described above. The term “treating” is defined as administration of a substance to a subject with the purpose to cure, alleviate, relieve, remedy, prevent, or ameliorate a disorder, symptoms of the disorder, a disease state secondary to the disorder, or predisposition toward the disorder. A “subject,” as used herein, refers to human and non-human animals, including all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human. In another embodiment, the subject is an experimental animal or animal suitable as a disease model. Identification of a candidate subject can be in the judgment of the subject or a health care professional, and can be subjective (e.g., opinion) or objective (e.g., measurable by a test or diagnostic method). An “effective amount” is an amount of the composition that is capable of producing a medically desirable result in a treated subject. The medically desirable result may be objective (i.e., measurable by some test or marker, e.g., decreased or increased expression of a gene) or subjective (i.e., subject gives an indication of or feels an effect). The treatment methods can be performed alone or in conjunction with other drugs and/or therapies.

In one in vivo approach, a composition containing an adiponectin variant of the invention is administered to a subject. Generally, the ccomposition is administered orally, by intravenous (i.v.) infusion, or injected or implanted subcutaneously, intramuscularly, intrathecally, intraperitoneally, intrarectally, intravaginally, intranasally, intragastrically, intratracheally, or intrapulmonarily. The dosage required depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

In some embodiments, polynucleotides such as DNA and RNA are administered to a subject. Polynucleotides can be delivered to target cells by, for example, the use of polymeric, biodegradable microparticle or microcapsule devices known in the art. Another way to achieve uptake of the nucleic acid is using liposomes, prepared by standard methods. The polynucleotides can be incorporated alone into these delivery vehicles or co-incorporated with tissue-specific or tumor-specific antibodies. Alternatively, one can prepare a molecular conjugate composed of a polynucleotide attached to poly-L-lysine by electrostatic or covalent forces. Poly-L-lysine binds to a ligand that can bind to a receptor on target cells. “Naked DNA” (i.e., without a delivery vehicle) can also be delivered to an intramuscular, intradermal, or subcutaneous site. A preferred dosage for administration of a polynucleotide is from approximately 10⁶ to 10¹² copies of the polynucleotide molecule.

In the relevant polynucleotides (e.g., expression vectors), the nucleic acid sequence encoding a sense or an antisense RNA is operatively linked to a promoter or enhancer-promoter combination. Suitable expression vectors include plasmids and viral vectors such as herpes viruses, retroviruses, vaccinia viruses, attenuated vaccinia viruses, canary pox viruses, adenoviruses and adeno-associated viruses, among others.

In a preferred embodiment adiponectin, or globular adiponectin, or variants of either full length or globular adiponectin would be used either alone or in combination therapy for the treatment of metabolic diseases including but not limited to obesity and the metabolic syndrome (Moller and Kaufman (2005) Ann. Rev. Med. 56:45-62, entirely incorporated by reference). Accordingly, the adiponectin variants of the present invention can be used to treat obesity, insulin resistance, glucose intolerance, hypertension, dyslipidemia (hypertriglyceridemia, and low HDL cholesterol levels), coronary heart diseases, and diabetes. Additionally, in this therapeutic mode, adiponectin or globular adiponectin could be used in combination with the following substances: insulin or insulin analogues, PPAR-agonists including but not limited to the TZD or fibrate classes of drugs, any member of the sulfonylurea class of drugs, the insulin-sensitizer metformin, GLP-1 antagonist drugs, or appetite suppressive agents such as orlistat, rimonobant, or other satiety inducing substances. The combination of adiponectin and any of these additional substances may improve the therapeutic effect of both drugs, especially the combination therapy with insulin.

The following examples are intended to illustrate, but not to limit, the scope of the invention. These examples are not meant to constrain the present invention to any particular application or theory of operation. While such examples are typical of those that might be used, other procedures known to those skilled in the art may alternatively be utilized. Indeed, those of ordinary skill in the art can readily envision and produce further embodiments, based on the teachings herein, without undue experimentation. For all positions discussed in the present invention, numbering is according to full length human adiponectin, as listed in FIG. 1.

### EXAMPLE 1

### Homology Modeling of Ad Collagen Region

The crystal structure of collagen (Protein Data Bank entry 1K6F) was used as a template to create the model of the trimeric human Ad collagen region required for subsequent calculations. Methods well known in the art were used to generate the human homology model.

### EXAMPLE 2

### Identification of Exposed Hydrophobic Residues in Ad Collagen Region

The Ad collagen region structure was analyzed to identify solvent-exposed hydrophobic residue. The absolute and fractional solvent-exposed hydrophobic surface area of each residue of each chain was calculated using the method of Lee and Richards ((1971) J. Mol. Biol. 55:379-400, entirely incorporated by reference) using an add-on radius of 1.4 Å (Angstroms). The values averaged over all three chains are listed in Table 1.

TABLE 1
Exposed Hydrophobic Residues in Ad Collagen Region
and Alternative Polar Residues

| | | Accessible | | |
|---|---|---|---|---|
| Residue # | WT | Surface Area | HEI | Alternative Polar Residues |
| 43 | ILE | 89.59 | 0.891 | D, E, H, K, N, Q, R, S |
| 53 | PRO | 83.50 | 0.410 | D, E, H, K, N, Q, R, S |
| 73 | LEU | 119.19 | 1.049 | D, E, H, K, N, Q, R, S |
| 74 | ILE | 73.07 | 0.727 | D, E, H, K, N, Q, R, S |
| 76 | PRO | 83.75 | 0.411 | D, E, H, K, N, Q, R, S |
| 80 | ILE | 100.80 | 1.002 | D, E, H, K, N, Q, R, S |
| 85 | VAL | 91.22 | 0.677 | D, E, H, K, N, Q, R, S |
| 94 | PHE | 110.30 | 0.886 | D, E, H, K, N, Q, R, S |
| 97 | ILE | 100.49 | 0.999 | D, E, H, K, N, Q, R, S |

A hydrophobicity exposure index (HEI) for each residue was calculated by multiplying the residue's fractional solvent-exposure by the Fauchere and Pliska hydrophobicity index for that amino acid residue type (Fauchere and Pliska (1983) Eur. J. Med. Chem. 18:369-75, entirely incorporated by reference) and listed in Table 1.

Solvent exposed hydrophobic residues in the Ad collagen region were defined to be hydrophobic residues with at least 50 Å² (square Angstroms) exposed hydrophobic surface area and HEI values greater than 0.4.

### EXAMPLE 3

### Identification of Alternative Polar Residues Based on Ad Ortholog Alignment

Orthologous Ad sequences from mouse (Genbank accession No. Q60994), rat (Genbank accession No. NP653345), rhesus maqaque (Genbank accession No. AAK92202), dog (Genbank accession No. NP001006645), boar (Genbank accession No. NP999535), cow (Genbank accession No. NP777167), and chicken (Genbank accession No. AAV48534) were obtained from NCBI, aligned to the human sequence (Genbank accession No. Q15848, SEQ ID NO:1) using the ClustalW algorithm (Higgins et al. (1994) Nucleic Acids Res. 22:4673-80, entirely incorporated by reference) and illustrated in FIG. 3. All alternative amino acid types present among these species at residue numbers 43-97 of SEQ ID NO:1 are listed in Table 2. From these, possible polar residues were identified.

TABLE 2
Alternative Polar Residues from Ortholog Alignment

| Residue # | WT | Ortholog Residues | Alternative Polar Residues |
|---|---|---|---|
| 43 | ILE | ALA | None |
| 53 | PRO | None | None |
| 73 | LEU | VAL | None |
| 74 | ILE | LEU, VAL, THR, GLN | THR, GLN |
| 76 | PRO | VAL | None |
| 80 | ILE | THR, PRO | THR |
| 85 | VAL | MET, ALA | None |
| 94 | PHE | None | None |
| 97 | ILE | THR, HIS | THR, HIS |

### EXAMPLE 4

### Identification of Regions of High Electrostatic Potential in Ad Collagen Region

The local electrostatic environment around each amino acid can contribute to the overall stability of the protein. Ideally, stability is conferred, for example, if negatively charged amino acids (e.g., aspartate at neutral pH) lie in areas of positive electrostatic potential and visa versa. Should, for example, an aspartate residue lie in a local environment of negative potential, substituting it with either a positively charged residue or a neutral polar residue may favorably stabilize the protein. This substitution, of course, depends on many structural factors for which the PDA® technology can account. Examining areas of high electrostatic potential may point to regions of the protein requiring optimal residue substitutions to improve overall protein stability.

The electrostatic potential at each position in the Ad collagen region was determined using the Debye-Huckel equation in the context of the Ad collagen region trimer. Positions in any of the three chains with electrostatic potential greater than 0.5 or less than −0.5 are listed in Table 3; modifications at these positions may confer increased stability or receptor binding specificity. Compensating mutations are unnecessary at positions for which the electrostatic potential and the charge of the wild-type amino acid are in agreement; this information is recorded in Table 3.

TABLE 3
Regions of High Electrostatic Potential in Ad Collagen
Region and Compensating Substitutions

| | | Electrostatic Potential | | | Compensating |
|---|---|---|---|---|---|
| Residue # | WT | Chain A | Chain B | Chain C | Substitutions |
| 55 | ARG | −0.74 | −0.86 | −0.25 | Not needed |
| 56 | ASP | 0.31 | 0.66 | 0.91 | Not needed |
| 57 | GLY | −0.94 | −0.88 | −0.50 | ARG, HIS, LYS |
| 58 | ARG | −1.01 | −0.61 | −0.64 | Not needed |
| 59 | ASP | 0.03 | 0.55 | 0.24 | Not needed |
| 60 | GLY | −0.66 | −0.75 | −1.00 | ARG, HIS, LYS |
| 61 | THR | −0.36 | −0.42 | −0.74 | ARG, HIS, LYS |
| 62 | PRO | −0.58 | −0.37 | −0.48 | ARG, HIS, LYS |
| 63 | GLY | −0.51 | −0.54 | −0.61 | ARG, HIS, LYS |
| 65 | LYS | −1.02 | −0.93 | −0.90 | Not needed |
| 66 | GLY | 0.09 | −0.11 | −0.56 | ARG, HIS, LYS |
| 67 | GLU | 0.41 | 0.58 | 0.36 | Not needed |
| 68 | LYS | −0.82 | −1.19 | −1.09 | Not needed |
| 70 | ASP | 0.30 | 0.30 | 0.54 | Not needed |
| 71 | PRO | −0.33 | −0.72 | −0.78 | ARG, HIS, LYS |
| 77 | LYS | −0.83 | −0.60 | −0.64 | Not needed |
| 80 | ILE | −0.78 | −0.91 | −0.77 | ARG, HIS, LYS |
| 81 | GLY | −0.93 | −0.92 | −0.73 | ARG, HIS, LYS |
| 83 | THR | −0.59 | −0.82 | −0.81 | ARG, HIS, LYS |
| 84 | GLY | −0.54 | −0.65 | −0.74 | ARG, HIS, LYS |
| 87 | GLY | −0.54 | −0.49 | −0.43 | ARG, HIS, LYS |
| 88 | ALA | −0.59 | −0.59 | −0.45 | ARG, HIS, LYS |
| 90 | GLY | −0.20 | −0.48 | −0.66 | ARG, HIS, LYS |
| 91 | PRO | 0.29 | −0.08 | −0.51 | ARG, HIS, LYS |
| 93 | GLY | 0.73 | 0.67 | 0.20 | ASP, GLU |
| 94 | PHE | 0.62 | 0.37 | 0.34 | ASP, GLU |
| 95 | PRO | 0.55 | 0.40 | 0.30 | ASP, GLU |
| 96 | GLY | 0.56 | 0.43 | 0.52 | ASP, GLU |
| 97 | ILE | 0.59 | 0.41 | 0.51 | ASP, GLU |
| 98 | GLN | 0.69 | 0.62 | 0.74 | ASP, GLU |
| 99 | GLY | 1.00 | 0.99 | 0.83 | ASP, GLU |
| 101 | LYS | −0.53 | −0.06 | 0.46 | Not needed |
| 102 | GLY | 0.09 | 0.40 | 0.61 | ASP, GLU |
| 103 | GLU | 0.17 | 0.33 | 0.64 | Not needed |
| 104 | PRO | −0.60 | −0.77 | −0.37 | ARG, HIS, LYS |
| 105 | GLY | −0.82 | −0.82 | −0.67 | ARG, HIS, LYS |
| 107 | GLY | −0.67 | −0.94 | −1.00 | ARG, HIS, LYS |
| 108 | ALA | −0.31 | −0.63 | −0.71 | ARG, HIS, LYS |

### EXAMPLE 5

### Replacement of Methionines in Ad to Improve Stability

While oxidation of manufactured protein therapeutics can be dependent on formulation and storage conditions (e.g., temperature and pH), the heterogeneity caused by oxidation can negatively impact clinical efficacy and safety. Ad contains methionine residues at positions 40, 128, 168, and 182. Removal may decrease formulation-dependent heterogeneity and improve storage stability. In a preferred embodiment, Ad MET residues are replaced by a group comprising of, but not limited to, ALA, ARG, ASN, ASP, GLN, GLU, HIS, ILE, LEU, LYS, SER, THR, or VAL.

### EXAMPLE 6

### Replacement of Hydroxyproline in Ad Collagen Region to Improve Bacterial

Collagen-related structural motifs have as their basis the amino acid sequence pattern of . . . [GXY] [GXY] . . . , where X and Y may be an amino or imino acid. Human collagens have a distinct preference for PRO at position Y. Typically a PRO at position Y is post-translationally modified through hydroxylation to hydroxyproline. In contrast, in bacterial collagens, the Y position is preferentially occupied by THR or GLN (Rasmussen et al. (2003) J. Biol. Chem. 278(34):32313-6, entirely incorporated by reference) instead of PRO, compensating for the lack of the hydroxylation reaction in bacteria. In Table 4, the hydroxyprolines in the Ad collagen region are listed, along with appropriate substitutions to improve bacterial expression, stability, and solubility or soluble expression.

TABLE 4
Hydroxyprolines in Ad Collagen Region and Appropriate Substitutions

| Residue # | WT | Appropriate Substitutions |
|---|---|---|
| 44 | PRO | THR, GLN |
| 47 | PRO | THR, GLN |
| 53 | PRO | THR, GLN |
| 62 | PRO | THR, GLN |
| 71 | PRO | THR, GLN |
| 86 | PRO | THR, GLN |
| 95 | PRO | THR, GLN |
| 104 | PRO | THR, GLN |

### EXAMPLE 7

### Replacement of [GXY] or [GXYGX′Y′] Repeat Units in Ad Collagen Region to Improve Stability

Host-guest experiments have found the following sequence motifs to be especially stabilizing in collagen: [GPR], [GER], [GPA], [GDR], [GKD], [GEK], [G_KGD_], [G_KGE_], [GE_G_K], [G_KG_E], [G_LGL_], [GL_GL_] (Persikov et al. (2005) J. Biol. Chem. 280(19):19343-9), where the “_” character represents a placeholder for any amino or imino acid. In a preferred embodiment, one or more amino acid replacements are made in the Ad collagen region to generate one or more of the stabilizing motifs listed.

### EXAMPLE 8

### Replacement of Aromatic Amino Acids in Non-Globular Ad to Improve Stability

It has been found that aromatic amino acids (F, H, W, Y) destabilize the collagen triple helix (Persikov et al. (2005) J. Biol. Chem. 280(19):19343-9, entirely incorporated by reference). In Table 5, the aromatic amino acids in the Ad collagen region are listed, along with appropriate substitutions to improve stability.

TABLE 5
Aromatic Amino Acids in Ad Collagen Region
and Appropriate Substitutions

| Residue # | WT | Appropriate Substitutions |
|---|---|---|
| 46 | HIS | PRO, ASP, GLU, LYS |
| 49 | HIS | PRO, ASP, GLU, LYS |
| 94 | PHE | PRO, ASP, GLU, LYS |

### EXAMPLE 9

### Especially Preferred Substitutions

In an especially preferred embodiment, amino acid substitutions are made from Table 6.

TABLE 6
Especially Preferred Substitutions in Ad Collagen Region

| Residue # | WT | Substitutions |
|---|---|---|
| 40 | MET | ALA, LEU |
| 43 | ILE | PRO, GLU |
| 44 | PRO | THR, GLN, ARG, LYS |
| 46 | HIS | PRO, ASP, GLU |
| 47 | PRO | THR, GLN, ARG, LYS |
| 49 | HIS | PRO |
| 53 | PRO | THR, GLN |
| 62 | PRO | THR, GLN, LYS |
| 71 | PRO | THR, GLN, ARG, LYS |
| 73 | LEU | PRO, ASP, GLU |
| 74 | ILE | THR, GLN, ARG, LYS |
| 80 | ILE | THR, GLN, ARG, LYS |
| 83 | THR | LYS |
| 85 | VAL | PRO |
| 86 | P | THR, GLN, ARG |
| 94 | F | PRO, ASP, GLU |
| 95 | P | THR, GLN, ARG, LYS |
| 97 | I | PRO, ASP, GLU |
| 104 | P | THR, GLN, ARG, LYS |

### EXAMPLE 10

### Homology Modeling of Ad Globular Region

The crystal structure of murine gAd (Protein Data Bank entry 1C3H, residues 111-247) was used as a template to create the human model required for subsequent PDA® library calculations as described above. FIG. 3 shows the sequence alignment between murine and human Ad sequences. No loop reconstruction was necessary since the alignment shows that no insertions or deletions exist between the globular domains of the two species. The PDA® algorithm was used to generate the human homology model.

### EXAMPLE 11

### Identification of Exposed Hydrophobic Residues in Ad Globular Region

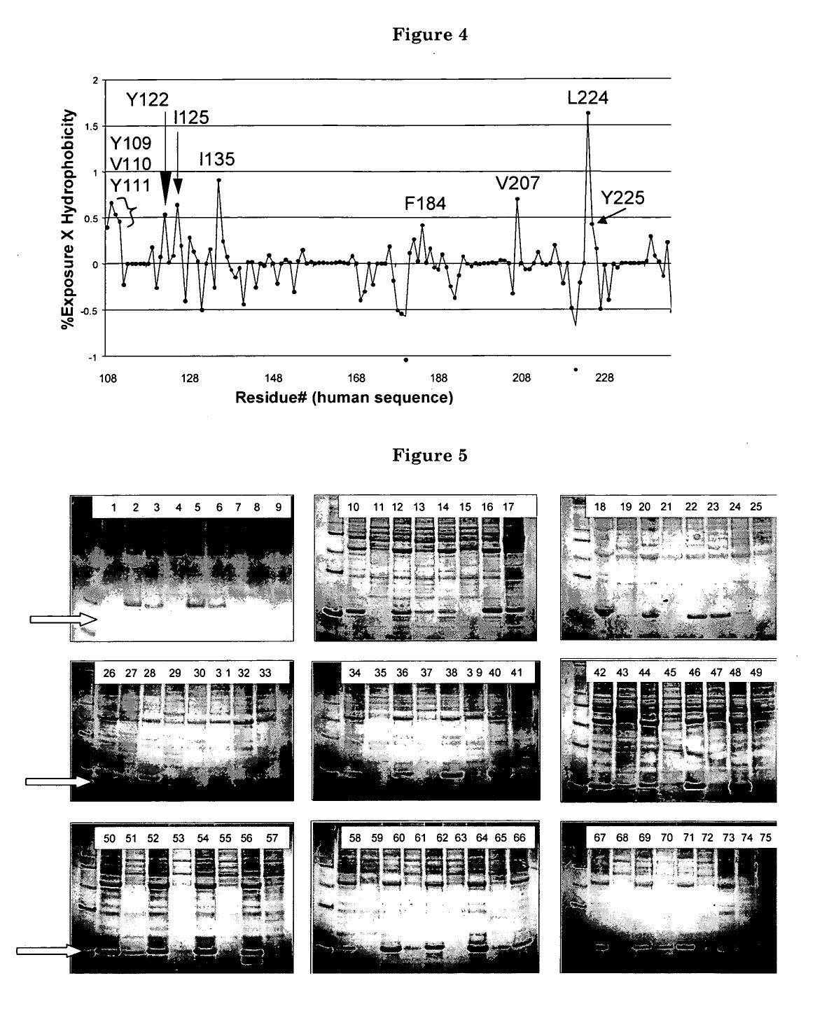

The gAd structure was analyzed to identify solvent-exposed hydrophobic residues. The absolute and fractional solvent-exposed hydrophobic surface area of each residue of each chain was calculated using the method of Lee and Richards ((1971) J. Mol. Biol. 55:379-400, entirely incorporated by reference) using an add-on radius of 1.4 Å (Angstroms). The values averaged over all three chains are listed in Table 7. FIG. 4 summarizes the HEI for each position in the gAd structure. Table 8 lists a subset of surface exposed hydrophobic amino acids having the highest HEI values and suggested alternative polar residues for each.

TABLE 7
Exposed Hydrophobic Residues in gAd

| | | Accessible | |
|---|---|---|---|
| Residue # | WT | Surface Area | HEI |
| 109 | TYR | 163.4 | 0.66 |
| 110 | VAL | 72.2 | 0.54 |
| 111 | TYR | 112.7 | 0.46 |
| 122 | TYR | 131.9 | 0.53 |
| 125 | ILE | 64.3 | 0.64 |
| 135 | ILE | 91.3 | 0.91 |
| 184 | PHE | 50.8 | 0.41 |
| 207 | VAL | 93.6 | 0.69 |
| 224 | LEU | 184.7 | 1.63 |
| 225 | TYR | 104.6 | 0.42 |

TABLE 8
Exposed Hydrophobic Residues in Ad Globular
Region and Alternative Polar Residues

| | | Alternative Polar Residues |
|---|---|---|
| Residue # | WT | (ΔE <2 kcal/mol) |
| 109 | Y | D, E, H, K, N, Q, R |
| 110 | V | D, E, H, K, N, Q, R, S |
| 111 | Y | D, E, H, K, N, Q, R |
| 122 | Y | D, E, H, N, R, S |
| 125 | I | D, E, H, K, N, Q, R, S |
| 135 | I | D, E, H, K, N, Q, R |
| 184 | F | D, H, K, N, R |
| 207 | V | D, E, H, K, N, Q, R, S |
| 224 | L | D, E, H, K, N, Q, R, S |
| 225 | Y | D, E, H, K, N, Q, R, S |

A hydrophobicity exposure index (HEI) for each residue was calculated as described in Example 2 and are also listed in Table 7. In order to identify positions most likely to impact solubility or soluble expression, solvent exposed hydrophobic residues in human gAd were defined to be hydrophobic residues with at least 50 Å² (square Angstroms) exposed hydrophobic surface area and HEI values greater than 0.4.

### EXAMPLE 12

### Identification of Alternative Polar Residues Based on Ad Ortholog Alignment

Orthologous Ad sequences were aligned to the human sequence (Genbank accession No. Q15848, SEQ ID NO:1) as described in Example 3. All alternative amino acid types present among these species at residue numbers 109-225 of SEQ ID NO:1 are listed in Table 9. From these, possible polar residues were identified.

TABLE 9
Alternative Polar Residues from Ortholog Alignment

| | | Ortholog | Alternative |
|---|---|---|---|
| Residue # | WT | Residues | Polar Residue |
| 109 | TYR | TYR | None |
| 110 | VAL | MET, VAL | None |
| 111 | TYR | HIS, TYR | HIS |
| 122 | TYR | TYR, ARG | ARG |
| 125 | ILE | HIS, ILE, VAL | HIS |
| 135 | ILE | ILE | None |
| 184 | PHE | PHE | None |
| 207 | VAL | LEU, LYS, VAL | LYS |
| 224 | LEU | ILE, LEU, VAL | None |
| 225 | TYR | TYR | None |

### EXAMPLE 13

### Identification of Preferred Substitutions to Ad to Improve Solubility or Soluble Expression

PDA® technology calculations were performed to identify alternate residues that are compatible with the structure of human Ad. At each variable position, energies were calculated for the wild type residue and alternate residues with decreased hydrophobic or increased polar character. Calculations were run using the homology-derived human gAd trimer created in Example 10.

First, point mutation calculations were run for the model along each monomer chain independently; no trimer symmetry was imposed to constrain identical rank orders of amino acids. The energy of each alternate amino acid in its most favorable rotameric conformation was compared to the energy of the wild type residue in the crystallographically observed rotameric conformation; all reported energies in Table 10 below are [E(lowest energy variant)-E(subsequent variant)]. In some cases, the wild type residue does not display the lowest energy. Since wt residues at these positions are surface-exposed hydrophobic amino acids and are presumably energetically destabilizing, this result is not surprising. Only polar amino acids exhibiting energies within 2.0 kcal/mol of the lowest energy amino acid are listed in Table 10. Results from all three trimer chains are listed and combined into a preferred list of alternative polar residues in Table 11. In a preferred embodiment, these substitutions are applied at single positions. In a more preferred embodiment, substitutions are simultaneously made at multiple positions. Coupling of energies for substitutions made at positions close together in three-dimensional space, however, could restrict some combinations of simultaneous substitutions.

TABLE 10
Energies of Most Favorable Polar Substitutions for
gAd Solvent-exposed Hydrophobic Positions

| | | Chain A | Chain B | Chain C |
|---|---|---|---|---|
| 109 | TYR | H: 0.33 | D: 1.71 | D: 1.86 |
| | | K: 1.56 | E: 1.95 | E: 1.92 |
| | | N: 1.89 | H: 0.49 | H: 0.52 |
| | | Q: 0.56 | K: 1.75 | N: 1.72 |
| | | R: 0.80 | N: 1.47 | Q: 0.57 |
| | | | Q: 0.36 | R: 1.67 |
| | | | R: 1.36 | |
| 110 | VAL | D: 0.80 | D: 0.00 | D: 0.00 |
| | | E: 1.22 | E: 0.29 | E: 0.27 |
| | | H: 0.43 | H: 0.85 | H: 0.78 |
| | | K: 0.27 | K: 1.19 | K: 1.59 |
| | | N: 0.72 | N: 1.39 | N: 1.30 |
| | | Q: 0.00 | Q: 0.61 | Q: 0.16 |
| | | R: 0.96 | R: 1.23 | R: 1.70 |
| | | S: 1.44 | | S: 1.83 |
| 111 | TYR | H: 0.96 | D: 1.44 | H: 0.37 |
| | | | E: 1.00 | N: 1.53 |
| | | | H: 0.18 | R: 1.56 |
| | | | K: 1.84 | |
| | | | N: 1.72 | |
| | | | Q: 1.02 | |
| | | | R: 1.48 | |
| 122 | TYR | H: 1.77 | D: 1.4 | E: 1.77 |
| | | | H: 1.51 | H: 1.43 |
| | | | N: 1.73 | N: 1.93 |
| | | | S: 1.42 | R: 1.87 |
| | | | | S: 1.85 |
| 125 | ILE | D: 0.63 | D: 1.34 | E: 0.00 |
| | | E: 0.00 | E: 0.00 | H: 1.46 |
| | | H: 0.25 | H: 0.83 | K: 1.92 |
| | | K: 0.78 | N: 1.30 | N: 1.76 |
| | | N: 0.45 | Q: 1.12 | Q: 1.63 |
| | | Q: 0.03 | R: 1.84 | |
| | | R: 0.49 | | |
| | | S: 1.43 | | |
| 135 | ILE | D: 1.69 | D: 1.34 | D: 1.61 |
| | | E: 0.16 | E: 0.70 | E: 0.38 |
| | | H: 0.29 | H: 0.48 | H: 0.39 |
| | | K: 1.61 | K: 1.15 | K: 0.88 |
| | | N: 1.57 | N: 1.61 | N: 1.72 |
| | | Q: 0.63 | Q: 0.57 | Q: 0.91 |
| | | R: 1.02 | R: 1.26 | R: 0.65 |
| 184 | PHE | D: 1.68 | H: 0.00 | K: 1.70 |
| | | H: 0.85 | K: 0.74 | R: 0.00 |
| | | N: 0.92 | N: 1.28 | |
| | | R: 2.00 | R: 1.35 | |
| 207 | VAL | D: 0.99 | D: 0.61 | D: 1.32 |
| | | E: 1.03 | E: 0.68 | E: 1.06 |
| | | H: 1.38 | H: 0.32 | H: 1.34 |
| | | K: 1.03 | K: 0.39 | K: 1.10 |
| | | N: 0.84 | N: 0.32 | N: 1.00 |
| | | Q: 0.09 | Q: 0.00 | Q: 0.00 |
| | | R: 0.69 | R: 0.19 | R: 0.30 |
| | | S: 1.87 | S: 1.74 | |
| 224 | LEU | D: 1.39 | D: 1.13 | D: 1.47 |
| | | E: 1.23 | E: 0.89 | E: 1.25 |
| | | H: 0.00 | H: 0.01 | H: 0.00 |
| | | K: 0.93 | K: 0.68 | K: 1.04 |
| | | N: 0.87 | N: 0.56 | N: 0.87 |
| | | Q: 0.52 | Q: 0.13 | Q: 0.57 |
| | | R: 0.20 | R: 0.00 | R: 0.22 |
| | | S: 1.78 | S: 1.48 | S: 1.86 |
| 225 | TYR | D: 0.87 | D: 0.99 | D: 0.00 |
| | | E: 1.50 | E: 1.91 | E: 0.98 |
| | | H: 0.61 | H: 1.37 | H: 0.18 |
| | | K: 0.99 | K: 1.76 | K: 0.67 |
| | | N: 0.53 | N: 0.74 | N: 0.14 |
| | | Q: 1.18 | Q: 1.66 | Q: 0.88 |
| | | R: 0.60 | R: 1.44 | R: 0.80 |
| | | S: 0.70 | S: 0.96 | S: 0.95 |

TABLE 11
Alternative Polar Residues

| Residue # | WT | Alternative Polar Residues |
|---|---|---|
| 109 | Y | D, E, H, K, N, Q, R |
| 110 | V | D, E, H, K, N, Q, R, S |
| 111 | Y | D, E, H, K, N, Q, R |
| 122 | Y | D, E, H, N, R, S |
| 125 | I | D, E, H, K, N, Q, R, S |
| 135 | I | D, E, H, K, N, Q, R |
| 184 | F | D, H, K, N, R |
| 207 | V | D, E, H, K, N, Q, R, S |
| 224 | L | D, E, H, K, N, Q, R, S |
| 225 | Y | D, E, H, K, N, Q, R, S |

### EXAMPLE 14

### Identification of Regions of High Electrostatic Potential in gAd

The electrostatic potential at each position in gAd was determined using the Debye-Huckel equation in the context of the gAd trimer. Positions in any of the three chains with electrostatic potential greater than 0.5 or less than −0.5 are listed in Table 12; modifications at these positions may confer increased stability or receptor binding specificity. In a preferred embodiment, D227 and D229 (average potentials of −0.5 and −0.6, respectively) are replaced with more preferred, positively charged amino acids. The PDA® technology was used to rank substituting D227 and D229 with either ARG, HIS (positively charged assuming formulation is below histidine's pKa of approximately 6.0) or LYS. The energy of each alternate positively charged amino acid in its most favorable rotameric conformation was compared to the energy of the most energetically favored residue; all reported energies in Table 13 are [E(lowest energy variant)-E(subsequent variant)]. All reported energies are within 1.5 kcal/mol of the lowest energy amino acid. In a preferred embodiment, D227 and/or D229 are substituted by a group comprising of, but not limited to, ARG, HIS and LYS.

TABLE 12
Regions of High Electrostatic Potential in gAd

| | | Electrostatic Potential | | |
|---|---|---|---|---|
| Residue | Residue | | | Chain |
| Number | Name | Chain A | Chain B | C |
| 110 | VAL | 0.67 | 0.60 | 0.61 |
| 129 | PRO | 0.49 | 0.53 | 0.48 |
| 134 | LYS | −0.84 | −0.88 | −0.85 |
| 144 | ASP | 0.45 | 0.49 | 0.53 |
| 165 | THR | −0.82 | −0.82 | −0.83 |
| 166 | VAL | −0.68 | −0.66 | −0.68 |
| 167 | TYR | −0.52 | −0.56 | −0.55 |
| 168 | MET | −0.60 | −0.59 | −0.59 |
| 169 | LYS | −0.61 | −0.49 | −0.46 |
| 171 | VAL | −1.00 | −0.94 | −0.99 |
| 172 | LYS | −1.14 | −1.07 | −1.17 |
| 173 | VAL | −0.76 | −0.70 | −0.74 |
| 182 | MET | 0.52 | 0.56 | 0.54 |
| 184 | PHE | −0.62 | −0.58 | −0.80 |
| 185 | THR | −0.91 | −0.89 | −0.89 |
| 186 | TYR | −0.83 | −0.71 | −0.87 |
| 187 | ASP | −0.81 | −0.70 | −0.79 |
| 188 | GLN | −0.55 | −0.32 | −0.75 |
| 189 | TYR | −1.41 | −1.32 | −1.37 |
| 190 | GLN | −1.13 | −1.08 | −1.22 |
| 192 | LYS | −0.73 | −0.47 | −0.49 |
| 194 | VAL | −0.41 | −0.58 | −0.52 |
| 195 | ASP | −0.79 | −0.72 | −0.79 |
| 196 | GLN | −1.06 | −1.07 | −1.03 |
| 197 | ALA | −1.22 | −1.19 | −1.19 |
| 204 | HIS | −0.51 | −0.50 | −0.52 |
| 208 | GLY | 0.51 | 0.38 | 0.36 |
| 209 | ASP | 0.67 | 0.70 | 0.67 |
| 210 | GLN | 0.72 | 0.74 | 0.74 |
| 212 | TRP | 0.49 | 0.51 | 0.50 |
| 222 | ASN | −0.44 | −0.50 | −0.35 |
| 227 | ASP | −0.45 | −0.47 | −0.54 |
| 229 | ASP | −0.61 | −0.59 | −0.64 |
| 230 | ASN | −0.59 | −0.58 | −0.60 |
| 240 | TYR | 0.55 | 0.54 | 0.54 |

TABLE 13
Energies of Most Favorable Positively Charged
Residues to Replace Aspartate 227 and 229 in gAd

| Residue | Residue | | | |
|---|---|---|---|---|
| Number | Name | Chain A | Chain B | Chain C |
| 227 | ASP | H: 0.00 | H: 1.54 | H: 0.48 |
| | | K: 0.90 | K: 0.00 | K: 0.00 |
| | | R: 1.32 | R: 0.81 | R: 0.30 |
| 229 | ASP | H: 0.66 | H: 0.00 | H: 0.52 |
| | | K: 1.27 | K: 0.18 | K: 0.67 |
| | | R: 0.50 | R: 0.37 | R: 0.66 |

### EXAMPLE 15

### Replacement of the Free Cysteine in gAd

The globular portion of Ad contains a single free cysteine at position 152. While C152 is not exposed to solvent in the crystal structure (the solvent accessible surface area averaged over all three chains is 1.1 Å²), the residue is located in an exterior loop and may be subject to local flexibility. In a preferred embodiment, removal of this cysteine may decrease non-specific disulfide formation and aggregation, and improve overall protein storage stability.

The energy of each alternate amino acid in its most favorable rotameric conformation was compared to the energy of the wild type cysteine residue; all reported energies in Table 14 are [E(CYS)-E(subsequent variant)]. In this case, the wild type residue does display the lowest energy. Only amino acids exhibiting energies within 5.0 kcal/mol of the lowest energy amino acid are listed. In a preferred embodiment, C152 is replaced by a group comprising of, but not limited to, ALA, ASN, SER, THR, and VAL.

TABLE 14
Energies of Most Favorable Non-glycine Residues
to Replace Cysteine 152 in Ad

| Residue | Residue | | | |
|---|---|---|---|---|
| Number | Name | Chain A | Chain B | Chain C |
| 152 | CYS | T: 1.78 | T: 1.13 | T: 1.22 |
| | | S: 2.08 | S: 2.39 | S: 1.78 |
| | | A: 3.56 | A: 3.85 | A: 3.32 |
| | | N: 4.89 | V: 4.20 | V: 4.78 |
| | | | N: 4.27 | |

### EXAMPLE 16

### Replacement of Methionines in gAd to Improve Stability

The globular portion of Ad contains three methionine residues (128, 168 and 182), two of which are exposed to solvent (128 and 182 with solvent accessible surface areas averaged over all three chains of 46.5 Å² and 43.7 Å², respectively) and may be prone to oxidation. Therefore, removal of these may decrease formulation-dependent heterogeneity and improve storage stability.

The energy of each alternate amino acid in its most favorable rotameric conformation was compared to the energy of the most energetically favored residue; all reported energies in Table 15 are [E(lowest energy variant)-E(subsequent variant)]. Only amino acids exhibiting energies within 4.0 kcal/mol of the lowest energy amino acid substitution are listed. In a preferred embodiment, MET 128 and 182 are replaced by a group comprising of, but not limited to, ALA, ARG, ASN, ASP, GLN, GLU, HIS, LYS, SER or THR.

TABLE 15
Energies of Most Favorable Non-glycine, Polar
Residues to Replace Methionine 128 and 182 in Ad

| Residue | Residue | | | |
|---|---|---|---|---|
| Number | Name | Chain A | Chain B | Chain C |
| 128 | MET | A: 1.52 | A: 1.21 | A: 1.22 |
| | | D: 1.03 | D: 1.04 | D: 1.06 |
| | | E: 1.46 | E: 0.83 | E: 0.92 |
| | | H: 0.00 | H: 0.06 | H: 0.00 |
| | | K: 1.96 | K: 2.70 | K: 1.80 |
| | | M: 0.80 | M: 1.85 | M: 1.61 |
| | | N: 0.98 | N: 1.32 | N: 0.99 |
| | | Q: 0.88 | Q: 1.01 | Q: 0.71 |
| | | R: 1.40 | R: 1.51 | R: 1.22 |
| | | S: 1.32 | S: 1.59 | S: 1.37 |
| | | T: 1.04 | T: 0.81 | T: 0.78 |
| 182 | MET | A: 2.18 | A: 2.66 | A: 2.22 |
| | | D: 3.79 | D: 3.60 | D: 2.23 |
| | | E: 0.00 | E: 2.18 | E: 1.68 |
| | | K: 3.48 | K: 3.16 | K: 3.31 |
| | | M: 0.37 | M: 2.13 | M: 2.40 |
| | | N: 3.45 | N: 2.45 | N: 3.12 |
| | | Q: 1.17 | Q: 2.66 | Q: 2.35 |
| | | R: 2.89 | R: 2.74 | R: 3.27 |
| | | S: 1.39 | S: 2.63 | S: 2.61 |
| | | T: 0.20 | T: 1.21 | T: 1.22 |

### EXAMPLE 17

### Identification of Preferred Coupled Substitutions to Ad to improve solubility or Soluble Expression

As discussed above, interaction energies for substitutions made at positions close together in three-dimensional space may restrict the identities of favorable amino acid combinations. In a preferred embodiment, positions comprising of the group of surface-exposed hydrophobic residues described in Example 11 and located within a sphere of 6 Å are identified and subjected to simultaneous design and optimization using the PDA® technology. Of positions 109, 110, 111, 122, 125, 135, 184, 207, 224, 225 described above, the following three groups are clusters of residues located within a 6 Å sphere of one another: 1) Y109, V110, and Y111, 2) Y122 and I125, and 3) L224 and Y225. The remaining positions (135, 184 and 207) are not located within 6 Å of any other surface-exposed hydrophobic residues identified in Example 11.

The energy of each alternate amino acid in its most favorable rotameric conformation was compared to the energy of the most energetically favored residue; all reported energies in Tables 16-18 are [E(lowest energy variant combination)-E(subsequent variant combination)]. Only polar amino acids were considered during the calculations and only amino acid combinations exhibiting energies within 2.0 kcal/mol of the lowest energy amino acid substitutions are listed. As in other examples, difference energies are listed for chains A, B and C. The residue combinations are sorted by the number of chains in which the listed substitution is energetically favored. In a preferred embodiment, substitution combinations are chosen that are energetically favorable in at least one of three chains. In a more preferred embodiment, substitutions are chosen that are favored in two of three chains. In a further preferred embodiment, substitutions are chosen that are favored in all three chains.

TABLE 16
Energies of Favored Coupled Substitutions at Positions 109, 110 and 111

| 109 | 110 | 111 | ΔE | | | 109 | 110 | 111 | ΔE | | |
|---|---|---|---|---|---|---|---|---|---|---|---|
| Y | V | Y | Chain A | Chain B | Chain C | Y | V | Y | Chain A | Chain B | Chain C |
| H | D | H | 1.14 | 0.23 | 0.38 | D | D | H | | 1.13 | |
| H | E | H | 1.55 | 0.43 | 0.83 | E | E | H | | 1.17 | |
| H | H | H | 1.02 | 0.71 | 1.08 | H | A | H | 1.64 | | |
| H | K | H | 1.12 | 1.16 | 1.20 | H | H | E | 1.65 | | |
| H | Q | H | 0.52 | 0.27 | 0.59 | H | K | E | 1.54 | | |
| H | R | H | 1.64 | 0.98 | 1.08 | H | N | H | 1.27 | | |
| H | T | H | 1.67 | 1.17 | 1.15 | K | D | E | | | 0.98 |
| Q | D | H | 1.51 | 0.33 | 0.57 | K | E | E | | | 0.88 |
| R | D | E | 1.50 | 1.16 | 1.01 | K | R | E | | | 0.70 |
| E | D | H | | 0.72 | 0.78 | K | T | E | | | 0.93 |
| E | Q | H | 1.38 | 1.00 | | N | D | H | | 1.06 | |
| H | Q | E | 0.99 | 0.95 | | Q | A | E | 1.52 | | |
| K | H | E | 1.17 | | 0.60 | Q | A | H | | 1.11 | |
| K | K | E | 1.17 | | 0.74 | Q | E | H | | 0.76 | |
| K | N | E | 1.54 | | 1.22 | Q | H | E | | | 1.13 |
| K | Q | E | 0.88 | | 0.45 | Q | K | H | 1.44 | | |
| Q | H | H | 1.33 | 1.12 | | Q | T | E | | | 1.10 |
| Q | Q | E | 1.40 | | 1.24 | R | N | E | 1.41 | | |
| Q | T | H | | 0.91 | 1.12 | R | Q | H | 1.70 | | |
| R | E | E | | 1.13 | 1.13 | | | | | | |
| R | H | E | 1.18 | | 0.96 | | | | | | |
| R | K | E | 1.25 | | 1.09 | | | | | | |
| R | Q | E | 0.80 | | 0.70 | | | | | | |
| R | R | E | 1.70 | | 0.93 | | | | | | |

TABLE 17
Energies of Favored Coupled Substitutions at Positions 122 and 125

| 122 | 125 | ΔE | | | 122 | 125 | ΔE | | |
|---|---|---|---|---|---|---|---|---|---|
| Y | I | Chain A | Chain B | Chain C | Y | I | Chain A | Chain B | Chain C |
| D | H | 1.60 | 0.81 | 1.17 | R | E | 0.22 | 0.16 | 0.35 |
| D | K | 0.84 | 0.35 | 1.21 | R | H | 1.48 | 1.16 | 1.13 |
| D | N | 1.70 | 0.89 | 1.60 | R | N | 1.59 | 1.38 | 1.44 |
| D | Q | 1.16 | 0.35 | 1.65 | R | Q | 1.08 | 0.80 | 1.40 |
| D | R | 0.78 | 0.71 | 1.17 | R | R | 1.46 | 1.62 | 1.80 |
| D | T | 1.67 | 0.91 | 1.36 | R | T | 1.57 | 1.30 | 1.26 |
| E | H | 1.77 | 0.46 | 0.00 | S | A | 1.55 | 0.59 | 1.02 |
| E | K | 1.62 | 1.12 | 0.93 | S | D | 1.26 | 0.31 | 1.42 |
| E | Q | 1.25 | 0.93 | 1.28 | S | E | 0.91 | 0.21 | 1.29 |
| E | R | 1.05 | 0.93 | 0.56 | S | H | 1.00 | 0.82 | 1.14 |
| H | A | 1.40 | 0.86 | 1.11 | S | N | 1.08 | 0.57 | 1.21 |
| H | D | 0.79 | 0.49 | 1.07 | S | Q | 0.59 | 0.00 | 1.37 |
| H | E | 0.00 | 0.28 | 0.47 | S | R | 0.92 | 0.76 | 1.43 |
| H | H | 0.85 | 0.81 | 0.78 | S | S | 1.78 | 0.95 | 1.41 |
| H | N | 0.82 | 0.89 | 1.29 | S | T | 1.08 | 0.51 | 1.04 |
| H | Q | 0.45 | 0.29 | 0.90 | D | A | | 0.93 | 1.33 |
| H | R | 0.81 | 1.07 | 1.61 | D | D | 1.98 | 0.81 | |
| H | S | 1.57 | 1.19 | 1.54 | D | E | 1.64 | 0.74 | |
| H | T | 0.91 | 0.77 | 1.08 | D | S | | 1.35 | 1.74 |
| K | A | 1.85 | 1.42 | 1.82 | E | A | | 1.51 | 1.35 |
| K | D | 1.38 | 0.12 | 1.33 | E | N | | 1.47 | 1.55 |
| K | E | 0.27 | 0.18 | 0.19 | E | S | | 1.83 | 1.72 |
| K | H | 1.28 | 1.54 | 1.70 | E | T | | 1.37 | 1.24 |
| K | N | 1.38 | 1.42 | 2.00 | H | K | 1.15 | 1.43 | |
| K | Q | 0.89 | 0.83 | 1.96 | K | R | 1.28 | 1.66 | |
| K | T | 1.37 | 1.32 | 1.83 | N | K | 1.54 | 1.48 | |
| N | A | 1.81 | 0.91 | 1.15 | N | S | | 1.29 | 1.55 |
| N | D | 1.55 | 0.67 | 1.57 | Q | A | | 1.40 | 1.43 |
| N | E | 0.97 | 0.57 | 1.32 | Q | K | 1.93 | 1.95 | |
| N | H | 1.28 | 1.10 | 1.30 | Q | S | | 1.76 | 1.85 |
| N | N | 1.37 | 0.90 | 1.37 | R | A | | 1.39 | 1.25 |
| N | Q | 0.85 | 0.33 | 1.52 | R | K | 1.81 | 1.98 | |
| N | R | 1.19 | 1.09 | 1.63 | R | S | | 1.74 | 1.66 |
| N | T | 1.36 | 0.86 | 1.18 | S | K | 1.27 | 1.11 | |
| Q | D | 1.78 | 1.09 | 1.79 | A | D | | 1.82 | |
| Q | E | 0.81 | 0.90 | 1.22 | A | E | | 1.73 | |
| Q | H | 1.63 | 1.54 | 1.52 | A | Q | | 1.53 | |
| Q | N | 1.70 | 1.39 | 1.62 | E | D | | 1.57 | |
| Q | Q | 0.76 | 0.81 | 1.66 | E | E | | 1.49 | |
| Q | R | 1.58 | 1.58 | 1.82 | K | K | 1.63 | | |
| Q | T | 1.71 | 1.31 | 1.45 | K | S | | 1.75 | |

TABLE 18
Energies of Favored Coupled Substitutions at Positions 224 and 225

| 224 | 225 | ΔE | | | 224 | 225 | ΔE | | |
|---|---|---|---|---|---|---|---|---|---|
| L | Y | Chain A | Chain B | Chain C | L | Y | Chain A | Chain B | Chain C |
| H | A | 1.35 | 0.22 | 1.51 | H | K | | 1.90 | |
| H | D | 1.15 | 0.00 | 1.22 | H | Q | | 1.20 | |
| H | E | 1.86 | 1.10 | 1.99 | H | R | | 1.61 | |
| H | H | 1.23 | 0.86 | 1.80 | K | A | | 1.22 | |
| H | N | 1.44 | 0.29 | 1.64 | K | D | | 0.66 | |
| H | S | 1.55 | 0.43 | 1.69 | K | N | | 0.97 | |
| H | T | 1.67 | 0.56 | 1.83 | K | Q | | 1.93 | |
| Q | D | 1.73 | 0.46 | 1.85 | K | S | | 1.27 | |
| Q | N | 1.63 | 0.41 | 1.93 | K | T | | 1.40 | |
| R | D | 1.65 | 0.28 | 1.90 | N | A | | 0.98 | |
| R | E | 1.41 | 0.59 | 1.62 | N | D | | 1.40 | |
| R | H | 1.17 | 0.81 | 1.76 | N | N | | 1.18 | |
| K | E | 1.87 | 0.89 | | N | R | | 1.90 | |
| K | H | 1.89 | 1.23 | | N | S | | 1.11 | |
| N | H | 1.78 | 1.80 | | N | T | | 1.26 | |
| Q | H | 1.48 | 1.10 | | Q | A | | 0.73 | |
| R | A | 1.95 | 0.13 | | Q | E | | 1.59 | |
| R | N | 1.83 | 0.46 | | Q | Q | | 1.48 | |
| R | S | 1.96 | 0.48 | | Q | R | | 1.27 | |
| D | A | | 1.59 | | Q | S | | 0.66 | |
| D | N | | 1.75 | | Q | T | | 0.70 | |
| D | S | | 1.65 | | R | K | | 1.56 | |
| D | T | | 1.76 | | R | Q | | 1.11 | |
| E | A | | 1.48 | | R | R | | 1.44 | |
| E | H | | 1.57 | | R | T | | 0.62 | |
| E | N | | 1.62 | | S | A | | 1.97 | |
| E | S | | 1.54 | | S | S | | 1.98 | |
| E | T | | 1.64 | | | | | | |

### EXAMPLE 18

### Core Design of gAd to Improve Stability

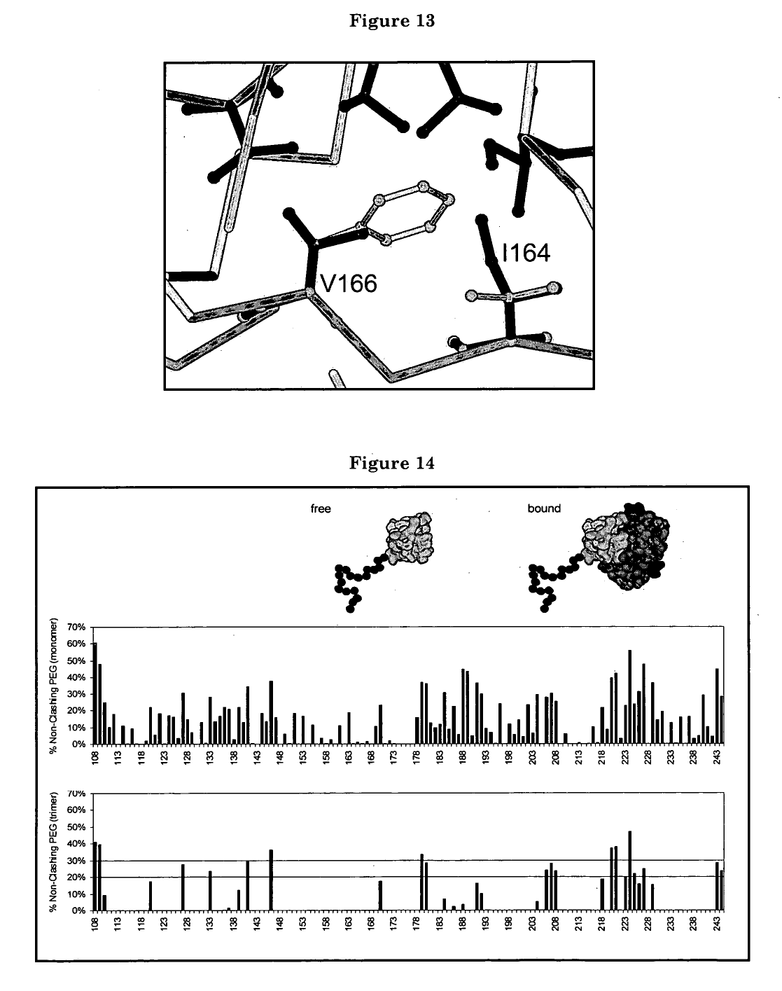

Optimization of packing interactions within the core of protein therapeutics has the potential to increase thermal stability, decrease aggregation, increase storage shelf-life and improve pharmacokinetics (Luo et al. (2002) Proteins 11:1218-26, entirely incorporated by reference). Buried hydrophobic residues (<5 Å² solvent accessible surface area averaged over all three chains) were identified as potential core residues. Hydrophobic residues located at the trimer interface were excluded from consideration. The first shell of buried core residues were defined as, but not limited to, F115, V123, 1130, F132, F150, F160, I164, V166, V171, V173, L175, L205, V211, L213, V215 and F234. These 16 residues were simultaneously subjected to optimization using the PDA® technology. Only substitutions with the following hydrophobic residues were considered: F, I, L, V and W. In a preferred embodiment, all non-polar amino acids are considered as energetically suitable substitutions. The top 100 sequence solutions are listed in Table 19 and are ranked by their energies relative the lowest energy sequence variant (E(lowest energy variant combination)-E(subsequent variant combination)). Solution #2 (1164V/V166F) is ˜2.5 kcal/mol lower in energy than the native sequence and is depicted in FIG. 13; substitution of V166 with PHE required losing a methyl group from position 164. In another preferred embodiment, additional buried residues could be included in the calculation such as residues V117, L119, I154 and L238. In another preferred embodiment, optimization can occur at single core positions or in combinations.

TABLE 19
Energies of Favored Substitutions at Core Positions within gAd

| | | 115 | 123 | 130 | 132 | 150 | 160 | 164 | 166 | 171 | 173 | 175 | 205 | 211 | 213 | 215 | 234 |
|---|---|---|---|---|---|---|---|---|---|---|---|---|---|---|---|---|---|
| WT | | F | V | I | F | F | F | I | V | V | V | L | L | V | L | V | F |
| 1 | 0.00 | — | — | V | — | — | — | V | F | — | — | — | — | — | — | — | — |
| 2 | 0.10 | — | — | — | — | — | — | V | F | — | — | — | — | — | — | — | — |
| 3 | 0.28 | — | — | V | — | — | — | V | F | — | — | — | — | — | I | — | — |
| 4 | 1.22 | — | I | V | — | — | — | — | — | — | — | — | — | — | — | — | — |
| 5 | 1.94 | — | — | — | — | — | — | V | F | — | — | — | — | — | V | — | — |
| 6 | 2.07 | — | I | — | — | — | — | — | — | — | — | — | — | — | — | — | — |
| 7 | 2.58 | — | — | — | — | — | — | — | — | — | — | — | — | — | — | — | — |
| 8 | 2.83 | — | — | V | — | — | — | — | — | — | — | — | — | — | — | — | — |
| 9 | 2.88 | — | I | V | — | — | — | — | — | — | — | — | — | — | I | — | — |
| 10 | 3.49 | — | — | — | — | — | — | V | F | — | — | — | — | — | I | — | — |
| 11 | 3.88 | — | I | V | — | — | — | V | — | — | — | — | — | — | — | — | — |
| 12 | 3.99 | — | — | V | — | — | — | V | — | — | — | — | — | — | — | — | — |
| 13 | 4.26 | — | I | V | — | — | — | V | — | — | — | — | — | — | I | — | — |
| 14 | 4.28 | — | — | V | — | — | — | V | F | — | — | — | — | — | V | — | — |
| 15 | 4.43 | — | I | — | — | — | — | V | — | — | — | — | — | — | — | — | — |
| 16 | 4.68 | — | I | V | — | — | — | V | — | — | — | — | — | — | V | — | — |
| 17 | 4.76 | — | — | V | — | — | — | — | F | — | — | — | — | — | V | — | — |
| 18 | 4.83 | — | I | V | — | — | — | — | — | — | — | — | — | — | — | I | — |
| 19 | 5.03 | — | I | V | — | — | — | — | — | — | — | — | — | — | V | — | — |
| 20 | 5.12 | — | I | — | — | — | — | — | — | — | — | — | — | — | I | — | — |
| 21 | 5.38 | — | — | V | — | — | — | — | — | — | — | — | — | — | I | — | — |
| 22 | 5.39 | — | — | — | — | — | — | V | — | — | — | — | — | — | — | — | — |
| 23 | 5.39 | — | — | V | — | — | — | V | — | — | — | — | — | — | I | — | — |
| 24 | 5.50 | — | — | V | — | — | — | — | — | — | — | — | — | — | — | I | — |
| 25 | 5.59 | — | — | V | — | — | — | — | — | — | — | — | — | — | V | — | — |
| 26 | 5.61 | — | I | V | — | — | — | — | — | — | — | V | — | — | — | — | — |
| 27 | 5.61 | — | I | V | — | — | — | — | — | — | — | V | — | — | I | — | — |
| 28 | 5.75 | — | — | V | — | — | — | — | F | — | — | — | — | — | — | — | — |
| 29 | 5.97 | — | — | — | — | — | — | L | — | — | — | — | — | — | — | — | — |
| 30 | 6.15 | — | — | V | — | — | — | V | — | — | — | — | — | — | V | — | — |
| 31 | 6.32 | — | — | — | — | L | — | V | F | — | — | — | — | — | — | — | — |
| 32 | 6.32 | — | — | V | — | — | — | V | — | — | — | — | — | — | — | I | — |
| 33 | 6.40 | — | — | — | — | — | — | — | — | — | — | — | — | — | V | — | — |
| 34 | 6.42 | — | — | V | — | — | — | — | — | — | — | V | — | — | — | — | — |
| 35 | 6.49 | — | — | V | — | — | — | — | F | — | — | — | — | — | I | — | — |
| 36 | 6.65 | — | — | V | — | — | — | — | — | — | — | V | — | — | I | — | — |
| 37 | 6.77 | — | — | V | — | — | — | L | — | — | — | — | — | — | — | — | — |
| 38 | 6.93 | — | I | — | — | — | — | — | — | — | — | — | — | — | — | I | — |
| 39 | 7.08 | — | — | V | — | L | — | V | F | — | — | — | — | — | — | — | — |
| 40 | 7.35 | — | — | V | — | — | — | V | — | — | — | — | — | — | I | I | — |
| 41 | 7.37 | — | — | — | — | — | — | — | F | — | — | — | — | — | — | — | — |
| 42 | 7.50 | I | — | V | — | — | — | V | F | — | — | — | — | — | I | — | — |
| 43 | 7.50 | — | I | V | — | L | — | — | — | — | — | — | — | — | — | — | — |
| 44 | 7.65 | L | — | V | — | — | — | V | F | — | — | — | — | — | I | — | — |
| 45 | 7.80 | — | — | V | — | — | — | L | — | — | — | — | — | — | — | I | — |
| 46 | 7.82 | — | — | V | — | — | — | L | — | — | — | — | — | — | I | — | — |
| 47 | 7.83 | — | — | L | — | — | — | — | — | — | — | — | — | — | — | — | — |
| 48 | 7.86 | — | — | — | — | — | — | V | F | — | — | V | — | — | — | — | — |
| 49 | 7.86 | — | I | V | — | — | — | L | — | — | — | — | — | — | V | — | — |
| 50 | 7.93 | — | — | — | — | — | L | V | F | — | — | — | — | — | — | — | — |
| 51 | 7.99 | — | — | V | — | — | — | L | — | — | — | — | — | — | V | — | — |
| 52 | 7.99 | — | — | V | — | — | — | V | — | — | — | — | — | — | V | I | — |
| 53 | 8.08 | — | I | — | — | L | — | — | — | — | — | — | — | — | — | — | — |
| 54 | 8.09 | — | — | — | — | — | — | — | — | — | — | — | — | — | — | I | — |
| 55 | 8.13 | — | — | V | — | — | — | — | — | — | — | — | — | — | I | I | — |
| 56 | 8.14 | I | — | V | — | — | — | V | F | — | — | — | — | — | — | — | — |
| 57 | 8.19 | — | — | V | — | — | — | L | — | — | — | — | — | — | I | I | — |
| 58 | 8.27 | V | — | V | — | — | — | V | F | — | — | — | — | — | — | — | — |
| 59 | 8.27 | — | — | V | — | L | — | V | F | — | — | — | — | — | I | — | — |
| 60 | 8.29 | — | — | — | — | L | — | V | F | — | — | — | — | — | I | — | — |
| 61 | 8.31 | I | — | V | — | — | — | V | F | — | — | — | — | — | V | — | — |
| 62 | 8.36 | — | I | V | — | L | — | — | — | — | — | — | — | — | I | — | — |
| 63 | 8.49 | — | I | V | — | — | — | — | — | — | — | — | — | — | I | I | — |
| 64 | 8.58 | — | — | — | — | L | — | — | — | — | — | — | — | — | — | — | — |
| 65 | 8.64 | — | — | V | — | L | — | — | — | — | — | — | — | — | — | — | — |
| 66 | 8.64 | — | I | V | — | — | L | — | — | — | — | — | — | — | — | — | — |
| 67 | 8.75 | — | I | V | — | — | — | V | — | — | — | V | — | — | I | — | — |
| 68 | 8.84 | — | I | — | — | — | — | L | — | — | — | — | — | — | — | — | — |
| 69 | 8.85 | — | — | — | — | — | I | — | — | — | — | — | — | — | — | — | — |
| 70 | 8.88 | — | I | V | — | — | I | — | — | — | — | — | — | — | — | — | — |
| 71 | 8.90 | — | I | — | — | — | — | V | — | — | — | — | — | — | — | I | — |
| 72 | 8.99 | — | I | V | — | L | — | V | — | — | — | — | — | — | — | — | — |
| 73 | 8.99 | — | — | V | — | — | I | V | F | — | — | — | — | — | I | — | — |
| 74 | 9.00 | V | — | V | — | — | — | V | F | — | — | — | — | — | I | — | — |
| 75 | 9.01 | — | I | V | — | L | — | V | — | — | — | — | — | — | I | — | — |
| 76 | 9.07 | — | I | V | — | — | — | — | — | — | — | V | — | — | V | — | — |
| 77 | 9.09 | — | — | V | — | — | — | — | — | — | — | V | — | — | V | — | — |
| 78 | 9.13 | — | — | — | — | — | — | — | — | — | — | — | — | — | I | — | — |
| 79 | 9.21 | — | — | V | — | — | V | V | F | — | — | — | — | — | I | — | — |
| 80 | 9.27 | V | — | V | — | — | — | V | F | — | — | — | — | — | V | — | — |
| 81 | 9.28 | W | I | V | — | — | V | V | — | — | — | — | — | — | — | — | — |
| 82 | 9.33 | L | I | — | — | — | — | — | — | — | — | — | — | — | — | — | — |
| 83 | 9.34 | — | I | V | — | — | — | V | — | — | — | F | — | — | — | — | — |
| 84 | 9.34 | — | — | — | — | — | — | V | — | — | — | — | — | — | I | — | — |
| 85 | 9.39 | — | — | V | — | L | — | — | — | — | — | — | — | — | I | — | — |
| 86 | 9.40 | — | — | V | — | — | — | V | F | — | — | V | — | — | I | — | — |
| 87 | 9.43 | — | I | V | — | — | — | V | — | — | — | F | — | — | I | — | — |
| 88 | 9.45 | — | — | — | — | — | — | V | — | — | — | — | — | — | V | — | — |
| 89 | 9.54 | — | I | V | — | — | — | L | — | — | — | — | — | — | — | — | — |
| 90 | 9.56 | W | I | V | — | — | I | V | — | — | — | — | — | — | — | — | — |
| 91 | 9.60 | — | — | — | — | — | — | V | F | — | — | F | — | — | V | — | — |
| 92 | 9.62 | — | — | V | — | L | — | V | — | — | — | — | — | — | I | — | — |
| 93 | 9.63 | — | I | V | — | — | — | V | — | — | — | — | — | — | — | I | — |
| 94 | 9.64 | I | — | V | — | — | — | — | F | — | — | — | — | — | I | — | — |
| 95 | 9.69 | I | — | V | — | — | — | — | F | — | — | — | — | — | V | — | — |
| 96 | 9.70 | — | I | — | — | L | — | V | — | — | — | — | — | — | — | — | — |
| 97 | 9.71 | — | — | — | — | L | — | V | — | — | — | — | — | — | — | — | — |
| 98 | 9.71 | — | I | V | — | — | I | — | — | — | — | — | — | — | I | — | — |
| 99 | 9.74 | — | I | V | — | — | — | V | — | — | — | — | — | — | I | I | — |
| 100 | 9.83 | — | I | V | — | L | — | — | — | — | — | — | — | — | V | — | — |

### EXAMPLE 19

### Rational PEGylation of gAd to Improve Pharmacokinetics and Pharmacodynamics

The methods of the present invention have been used to select optimal PEGylation sites in gAd (see FIG. 1) based on the atomic coordinates generated in Example 10. The A chain was focused on for the rational PEGylation analysis.

The simulation data was first analyzed to identify sites with high coupling efficiency. For PEG2000, sites for which greater than 20% of the simulated PEG chains are non-clashing in the free state are considered optimal sites for attachment (see FIG. 14, top chart). These sites include A108, Y109, V110, E120, N127, T133, F136, Y137, Q139, N141, S146, D170, D179, K180, F184, Y186, Q188, Y189, E191, K192, Q196, L202, H204, E206, V207, G208, D218, E220, R221, G223, L224, Y225, A226, D227, D229, Y240, T243, and N244.

The predicted high coupling efficiency sites were further screened to identify which of these sites retain PEG range of motion upon receptor binding. For PEG2000, sites for which greater than 20% of the simulated PEG chains are non-clashing in the bound state are preferred (see FIG. 14). These sites include A108, Y109, N127, T133, N141, S146, D179, K180, E206, V207, G208, E220, R221, G223, L224, Y225, D227, T243, and N244. For PEG2000, sites for which greater than 30% of the simulated PEG are not clashing in the bound state are especially preferred. These sites include A108, Y109, S146, D179, E220, R221, and L224.

In a preferred embodiment, site specific PEGylation at any of these or other positions would either require replacement of the native amino acid with a suitable amino acid such as cysteine or the introduction of an unnatural amino acid such as p-acetyl-L-phenylalanine.

In another preferred embodiment, a bivalent PEG could be used to form a link between two gAd molecules. This may replace the collagen-like domain and form a hexameric gAd unit of two trimeric gAd units.

### EXAMPLE 20

### Construction and Expression of Globular Adiponectin with Solubility or Soluble Expression Enhancing Amino Acid Substitutions

Standard molecular biology methods were employed to construct an expression library of globular adiponectin variants. Briefly, gAd cDNA (encoding amino acids 110-244) was subcloned into the bacterial expression vector pET-17b. Site directed mutagenesis was performed using standard methods to generate the 34 single amino acid substitution variants listed in Table 20.

TABLE 20

| Native | | Variant | | Variant |
|---|---|---|---|---|
| residue | Position | residue | Codon | name |
| Y | 122 | E | GAA | Y122E |
| Y | 122 | H | CAC | Y122H |
| Y | 122 | S | TCC | Y122S |
| I | 125 | E | GAA | I125E |
| I | 125 | H | CAC | I125H |
| I | 125 | R | CGC | I125R |
| I | 125 | T | ACC | I125T |
| I | 135 | E | GAA | I135E |
| I | 135 | H | CAC | I135H |
| I | 135 | Q | CAG | I135Q |
| I | 135 | R | CGC | I135R |
| I | 135 | T | ACC | I135T |
| F | 184 | E | GAA | F184E |
| F | 184 | H | CAC | F184H |
| F | 184 | R | CGC | F184R |
| F | 184 | T | ACC | F184T |
| V | 207 | A | GCT | V207A |
| V | 207 | E | GAA | V207E |
| V | 207 | K | AAA | V207K |
| V | 207 | Q | CAG | V207Q |
| V | 207 | T | ACC | V207T |
| L | 224 | E | GAA | L224E |
| L | 224 | H | CAC | L224H |
| L | 224 | Q | CAG | L224Q |
| L | 224 | R | CGC | L224R |
| L | 224 | S | TCC | L224S |
| Y | 225 | E | GAA | Y225E |
| Y | 225 | H | CAC | Y225H |
| Y | 225 | R | CGC | Y225R |
| Y | 225 | S | TCC | Y225S |
| D | 227 | R | CGC | D227R |
| D | 229 | R | CGC | D229R |
| Y | 122 | R | CGC | Y122R |

We used standard protein expression and analysis methods to express the single amino acid gAd variants listed in Table 20. Briefly, we generated a fresh lawn of colonies of gAd variants in BL21Star (DE3) cells and the entire lawn was harvested and used to inoculate a 50 mL starter culture for each clone. Cultures were grown at 37° C. until they reached an optical density (OD₆₀₀) of 0.6 in approximately 1.5 hours. The cultures were cooled to room temperature, induced with 0.5 mM IPTG, and grown for approximately additional hours in a shaker set to room temperature. The cultures were harvested, OD₆₀₀ was measured, and bacterial pellets were prepared by centrifugation at 6000 rpm for 15 minutes. The supernatant was discarded and the pellet was solubilized using BugBuster HT (a proprietary detergent-containing bacterial lysis reagent). Soluble and insoluble lysate fractions were analyzed by SDS-PAGE using standard electrophoresis methods.

FIG. 5 features nine SDS-PAGE gels that were loaded with equal amounts of the soluble and insoluble fractions of the 34 single amino acid substitution variants. SDS-PAGE loading is as shown in Table 21. Globular adiponectin is a 134 amino acid polypeptide with a molecular mass of ˜15 kD. In FIG. 5, gAd is highlighted by an arrow on the left hand margin.

TABLE 21
SDS-PAGE Loading to Screen the Soluble or
Insoluble Fractions of Library #1 Variants

| | | Fraction: |
|---|---|---|
| | | [S]oluble, |
| | | [I]nsoluble, or |
| Lane # | Variant | [T]otal |
| 1 | Y122E | T |
| 2 | Y122E | I |
| 3 | Y122E | S |
| 4 | I135H | T |
| 5 | I135H | I |
| 6 | I135H | S |
| 7 | V207D | T |
| 8 | V207D | I |
| 9 | V207D | S |
| 10 | L224R | I |
| 11 | L224R | S |
| 12 | NATIVE | I |
| 13 | NATIVE | S |
| 14 | I135Q | I |
| 15 | I135Q | S |
| 16 | V207E | I |
| 17 | V207E | S |
| 18 | L224S | I |
| 19 | L224S | S |
| 20 | Y122R | I |
| 21 | Y122R | S |
| 22 | Y122S | I |
| 23 | Y122S | S |
| 24 | I135R | I |
| 25 | I135R | S |
| 26 | V207K | I |
| 27 | V207K | S |
| 28 | Y225E | I |
| 29 | Y225E | S |
| 30 | pET-17b | I |
| 31 | pET-17b | S |
| 32 | I125E | I |
| 33 | I125E | S |
| 34 | I135T | I |
| 35 | I135T | S |
| 36 | V207Q | I |
| 37 | V207Q | S |
| 38 | Y225H | I |
| 39 | Y225H | S |
| 40 | NATIVE | I |
| 41 | NATIVE | S |
| 42 | I125H | I |
| 43 | I125H | S |
| 44 | F184E | I |
| 45 | F184E | S |
| 46 | V207T | I |
| 47 | V207T | S |
| 48 | Y225R | I |
| 49 | Y225R | S |
| 50 | I125T | I |
| 51 | I125T | S |
| 52 | F184R | I |
| 53 | F184R | S |
| 54 | L224H | I |
| 55 | L224H | S |
| 56 | D227R | I |
| 57 | D227R | S |
| 58 | I135E | I |
| 59 | I135E | S |
| 60 | F184T | I |
| 61 | F184T | S |
| 62 | L224Q | I |
| 63 | L224Q | S |
| 64 | D229R | I |
| 65 | D229R | S |
| 66 | Y122H | I |
| 67 | I125R | I |
| 68 | I125R | S |
| 69 | F184H | I |
| 70 | F184H | S |
| 71 | L224E | I |
| 72 | L224E | S |
| 73 | Y225S | I |
| 74 | Y225S | S |
| 75 | Y122H | S |

When gAd-expressing cells are lysed under these detergent-containing conditions (i.e,. BugBuster), the native gAd is found to be <10% soluble (FIG. 5, lanes 12-13 and 40-41). We identified several variants that had improved protein solubility or soluble expression under these expression and lysis conditions. Variants Y122H (FIG. 5, lanes 66 and 75), Y122S (FIG. 5, lanes 22-23), I125E (FIG. 5, lanes 32-33), I125H (FIG. 5, lanes 42-43), I125T (FIG. 5, lanes 50-51), F184H (FIG. 5, lanes 69-70), V207E (FIG. 5, lanes 16-17), and V207K (FIG. 5, lanes 26-27) all had solubility or soluble expression equal to or in many cases far greater than native gAd.

### EXAMPLE 21

### Solubility or Soluble Expression Analysis of Select Globular Adiponectin Single Amino Acid Substitution Variants in the Absence of Detergent

Variants Y122H, Y122S, I125E, I125H, I125T, F184H, V207E, and V207K were selected based on their improved solubility properties as judged from the pilot expression studies described above. In order to demonstrate that these variants have truly improved solubility, it was necessary to measure the amount of soluble protein generated when bacteria expressing these protein are lysed in the absence of detergent. Solubility in the absence of detergent is recognized a more rigorous measure of soluble protein and it enables future downstream process modifications and may lead to a streamlined manufacturing process.

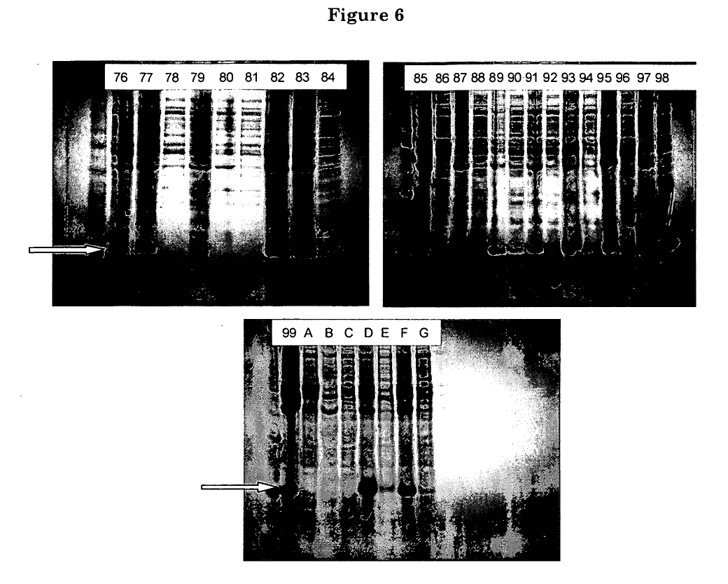

The variants were expressed as described above except that the vessel volume was scaled up ten-fold (500 mL in a 2000 mL flask). After overnight induction at 4° C., the cells were harvested by centrifugation and the pellets were stored at −80° C. The cell pellets were mixed with detergent-free lysis buffer (20 mM BisTris pH 6.0, 1 mM EDTA, 0.5 mM DTT) and lysed by sonic disruption. The resulting material was cleared by high-speed centrifugation, and the resulting cleared soluble and insoluble fractions were volume normalized and analyzed using SDS-PAGE. This approach allows the determination of the improvement of overall protein expression/yield as well as solubility. FIG. 6 shows three SDS-PAGE gels that contained the soluble and insoluble fractions of native gAd, empty vector (pET-17b), or the selected variants. The gels were loaded as described in Table 22; an arrow on the left hand margin of the figure points to the gAd controls.

TABLE 22
SDS-PAGE Loading to Screen the Soluble or Insoluble
Fractions of Select Variants in the Absence of Detergent

| | | Fraction: |
|---|---|---|
| | | [S]oluble, |
| | | [I]nsoluble, or |
| Lane # | Variant | [T]otal |
| 76 | NATIVE | T |
| 77 | NATIVE | I |
| 78 | NATIVE | S |
| 79 | pET-17b | T |
| 80 | pET-17b | I |
| 81 | pET-17b | S |
| 82 | I125E | T |
| 83 | I125E | I |
| 84 | I125E | S |
| 85 | NATIVE | I |
| 86 | NATIVE | S |
| 87 | pET-17b | I |
| 88 | pET-17b | S |
| 89 | Y122H | I |
| 90 | Y122H | S |
| 91 | Y122S | I |
| 92 | Y122S | S |
| 93 | I125H | I |
| 94 | I125H | S |
| 95 | I125T | I |
| 96 | I125T | S |
| 97 | F184H | I |
| 98 | F184H | S |
| 99 | NATIVE | I |
| A | NATIVE | S |
| B | PET-17b | I |
| C | PET-17b | S |
| D | V207E | I |
| E | V207E | S |
| F | V207K | I |
| G | V207K | S |

When the gAd-expressing cells were lysed under these detergent-free conditions, the native gAd was found to be virtually insoluble (FIG. 6, lanes 76-78, 85-86, and 99-A). All the variants tested had dramatically improved solubility in the absence of detergent. Especially favorable in this regard were the substitutions I125E, I125T, and Y122H. Furthermore, since these samples were volume normalized, we identified numerous variants with significantly improved protein expression yields. Variants F184H, I125H, and V207E had the greatest effect on increasing gAd protein yields.

### EXAMPLE 22

### Construction and Expression Analysis of Double Variant Globular Adiponectin Proteins

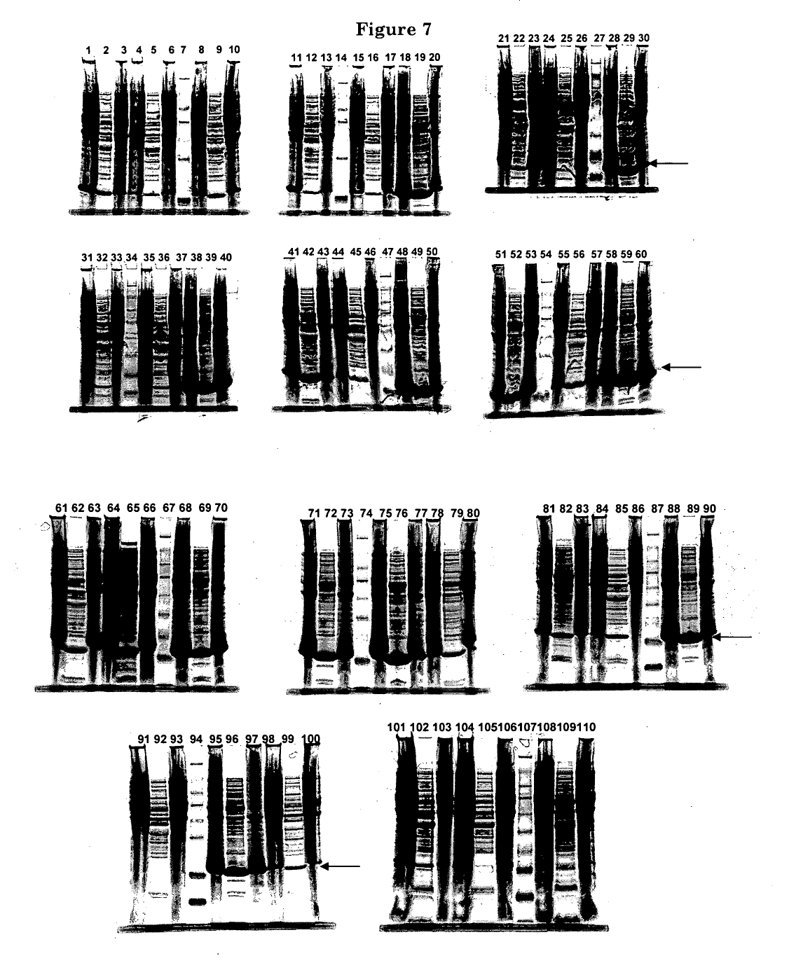

The eight globular adiponectin amino acid substitutions that gave increased solubility and expression yields were combined in pair wise combination to generate a library of adiponectin double variants. The same molecular biology techniques and codons as described above were used to generate the following double mutant globular adiponectin variants; F184H/Y122H, F184H/Y122S, F184H/I125E, F184H/I125H, F184H/I125T, F184H/V207E, F184H/V207K, V207E/Y122H, V207E/Y122S, V207E/I125E, V207E/I125H, V207E/I125T, V207K/Y122H, V207K/Y122S, V207K/I125E, V207K/I125H, V207K/I125T, I125E/Y122H, I125E/Y122S, I125H/Y122H, I125H/Y122S, I125T/Y122H, I125T/Y122S. These proteins were expressed and processes as described above in Example 20. After detergent-induced lysis, we compared the relative amount of soluble protein with the total and insoluble fractions. FIG. 7 shows 11 SDS-PAGE gels that contained the expression and solubility information for the double mutant globular adiponectin variants. As an experimental control, single mutants and native globular adiponectin were included, as well as an empty vector control. On the SDS-PAGE, an arrow highlights the position of globular adiponectin.

TABLE 23
SDS-PAGE Loading to Screen the Total, Soluble, and
Insoluble Fractions of Double Mutant Globular Adiponectin
Variants in the Presence of Detergent

| | | Fraction: |
|---|---|---|
| | | [S]oluble, |
| | | [I]nsoluble, |
| Lane # | Variant | or [T]otal |
| 1 | Y122H | T |
| 2 | Y122H | I |
| 3 | Y122H | S |
| 4 | Y122S | T |
| 5 | Y122S | I |
| 6 | Y122S | S |
| 7 | Marker | |
| 8 | I125E | T |
| 9 | I125E | I |
| 10 | I125E | S |
| 11 | I125H | T |
| 12 | I125H | I |
| 13 | I125H | S |
| 14 | Marker | |
| 15 | I125T | T |
| 16 | I125T | I |
| 17 | I125T | S |
| 18 | F184H | T |
| 19 | F184H | I |
| 20 | F184H | S |
| 21 | V207E | T |
| 22 | V207E | I |
| 23 | V207E | S |
| 24 | V207K | T |
| 25 | V207K | I |
| 26 | V207K | S |
| 27 | Marker | |
| 28 | F184H/Y122H | T |
| 29 | F184H/Y122H | I |
| 30 | F184H/Y122H | S |
| 31 | F184H/Y122S | T |
| 32 | F184H/Y122S | I |
| 33 | F184H/Y122S | S |
| 34 | Marker | |
| 35 | F184H/I125E | T |
| 36 | F184H/I125E | I |
| 37 | F184H/I125E | S |
| 38 | F184H/I125H | T |
| 39 | F184H/I125H | I |
| 40 | F184H/I125H | S |
| 41 | F184H/I125T | T |
| 42 | F184H/I125T | I |
| 43 | F184H/I125T | S |
| 44 | F184H/V207E | T |
| 45 | F184H/V207E | I |
| 46 | F184H/V207E | S |
| 47 | Marker | |
| 48 | F184H/V207K | T |
| 49 | F184H/V207K | I |
| 50 | F184H/V207K | S |
| 51 | V207E/Y122H | T |
| 52 | V207E/Y122H | I |
| 53 | V207E/Y122H | S |
| 54 | Marker | |
| 55 | V207E/Y122S | T |
| 56 | V207E/Y122S | I |
| 57 | V207E/Y122S | S |
| 58 | V207E/I125E | T |
| 59 | V207E/I125E | I |
| 60 | V207E/I125E | S |
| 61 | V207E/I125H | T |
| 62 | V207E/I125H | I |
| 63 | V207E/I125H | S |
| 64 | V207E/I125T | T |
| 65 | V207E/I125T | I |
| 66 | V207E/I125T | S |
| 67 | Marker | |
| 68 | V207K/Y122H | T |
| 69 | V207K/Y122H | I |
| 70 | V207K/Y122H | S |
| 71 | V207K/Y122S | T |
| 72 | V207K/Y122S | I |
| 73 | V207K/Y122S | S |
| 74 | Marker | |
| 75 | V207K/I125E | T |
| 76 | V207K/I125E | I |
| 77 | V207K/I125E | S |
| 78 | V207K/I125H | T |
| 79 | V207K/I125H | I |
| 80 | V207K/I125H | S |
| 81 | V207K/I125T | T |
| 82 | V207K/I125T | I |
| 83 | V207K/I125T | S |
| 84 | I125E/Y122H | T |
| 85 | I125E/Y122H | I |
| 86 | I125E/Y122H | S |
| 87 | Marker | |
| 88 | I125E/Y122S | T |
| 89 | I125E/Y122S | I |
| 90 | I125E/Y122S | S |
| 91 | I125H/Y122H | T |
| 92 | I125H/Y122H | I |
| 93 | I125H/Y122H | S |
| 94 | Marker | |
| 95 | I125H/Y122S | T |
| 96 | I125H/Y122S | I |
| 97 | I125H/Y122S | S |
| 98 | I125T/Y122H | T |
| 99 | I125T/Y122H | I |
| 100 | I125T/Y122H | S |
| 101 | I125T/Y122S | T |
| 102 | I125T/Y122S | I |
| 103 | I125T/Y122S | S |
| 104 | Native | T |
| 105 | Native | I |
| 106 | Native | S |
| 107 | Marker | |
| 108 | pET-17b | T |
| 109 | pET-17b | I |
| 110 | pET-17b | S |

Several of the double mutant proteins had dramatically improved expression and solubility properties. Of the 23 double variant proteins tested, variants F184H/Y122H, F184H/I125H, F184H/I125T, F184H/V207K, V207E/I125E, V207K/Y122S, V207K/I125E, and I125E/Y122S had the most dramatic improvements.

### EXAMPLE 23

### Solubility Analysis of Select Globular Adiponectin Double Amino Acid Substitution Variants in the Absence of Detergent

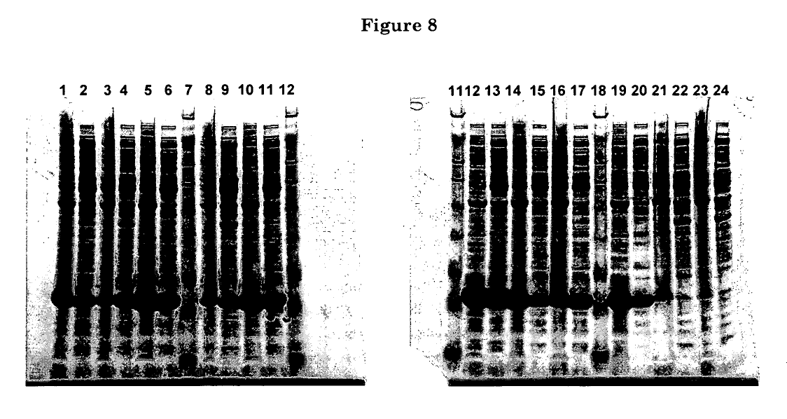

Variants F184H/Y122H, F184H/I125H, F184H/I125T, F184H/V207K, V207E/I125E, V207K/Y122S, V207K/I125E, and I125E/Y122S were subjected to the same protein solubility analysis as described in Example 21. FIG. 8 shows two SDS-PAGE gels that contained the results of the solubility analysis in the absence of detergent. Upon lysis of the bacteria by sonication, there is an increase of both total and soluble protein released for the gAd double variants when compared to the native protein. Table 24 shows the SDS-PAGE loading for the lysates prepared from the double variants and native proteins, the highest expressing single variant F184H was included as an additional control.

TABLE 24
SDS-PAGE Loading to Screen the Soluble or Insoluble Fractions
of Select Double Variants in the Absence of Detergent

| | | Fraction: |
|---|---|---|
| | | [S]oluble or |
| Lane # | Variant | [I]nsoluble |
| 1 | F184H | I |
| 2 | F184H | S |
| 3 | F184H/Y122H | I |
| 4 | F184H/Y122H | S |
| 5 | F184H/I125H | I |
| 6 | F184H/I125H | S |
| 7 | Marker | |
| 8 | F184H/I125T | I |
| 9 | F184H/I125T | S |
| 10 | F184H/V207K | I |
| 11 | F184H/V207K | S |
| 12 | V207E/I125E | I |
| 13 | V207E/I125E | S |
| 14 | V207K/Y122S | I |
| 15 | V207K/Y122S | S |
| 16 | V207K/I125E | I |
| 17 | V207K/I125E | S |
| 18 | Marker | |
| 19 | I125E/Y122S | I |
| 20 | I125E/Y122S | S |
| 21 | Native | I |
| 22 | Native | S |
| 23 | Native | I |
| 24 | Native | S |

The majority of these variants have a nearly equal partitioning of protein between the soluble and insoluble fractions, suggesting approximately 50% solubility. Variants F184/H/Y122H, F184H/I125T, and V207K/I125E appear to have even greater than 50% solubility. Finally, when compared to the native protein, there is a several orders of magnitude increase in the amount of total expressed and soluble globular adiponectin.

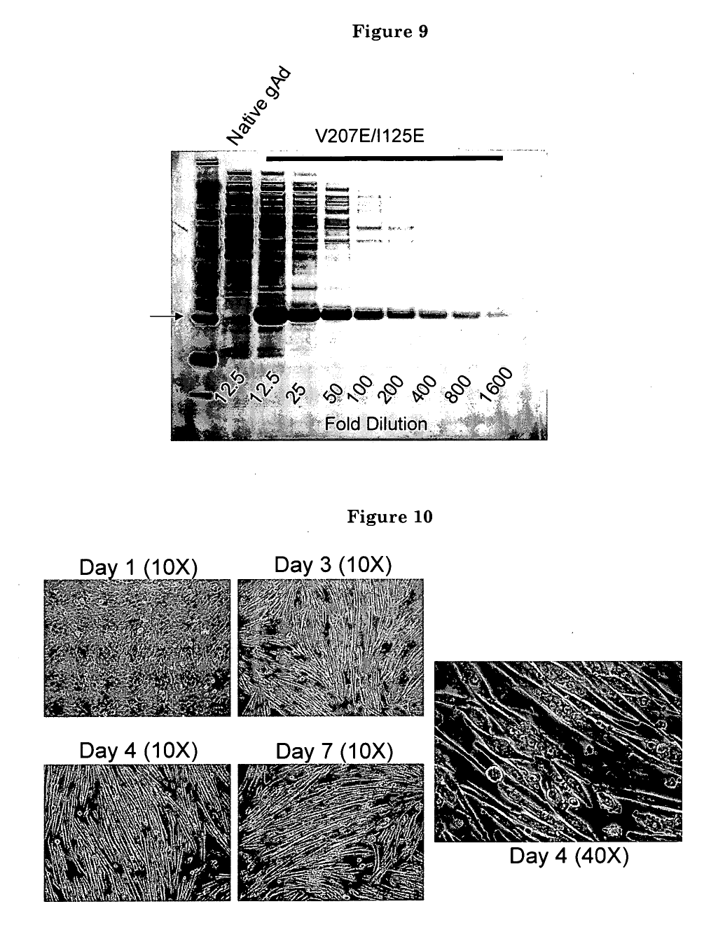

FIG. 9 shows an SDS-PAGE that contained the detergent-free soluble lysates from native and V207E/I125E gAd. The native lysate was diluted 12.5-fold and compared to an equal or serial dilution of the identical lysate made from E. coli expressing the V207E/I125E gAd variant. It is clear from this analysis that there is at least a 100-1000 fold difference in the amount of soluble protein generated by the V207E/I125E gAd variant relative to native gAd.

### EXAMPLE 24

### gAd Double Variants Induce AMPK Phoshorylation in Differentiated Mouse C2C12 Cells

To measure the biological activity of select gAd variants, it was necessary to purify the recombinant gAd proteins away for the E. coli host cell contaminants. We developed a conventional chromatography process that consisted of three separate column steps. Briefly, gAd variants were grown and processed into lysate as described in Example 20, the soluble fraction was applied to a DEAE column and eluted with an isocratic step at 200 mM NaCl. This material was passed over Q column as a non-binding step (i.e., the gAd flowed through the column but protein contaminants and endotoxin were bound), and finally polished using a preparative S-100HR gel filtration column. For the gAd variants this process would routinely yield 100-300 mg of purified protein per liter of E. coli culture.

We used C2C12 cells differentiated into myotubes to measure gAd-induced phosphorylation of AMP Kinase (AMPK). Murine C2C12 cells were grown in culture as described by the ATCC. Differentiation was induced by transferring the cells to a growth media containing 2% horse serum. The cells were maintained in this media for up to seven days. During this time, the cells elongated and fused together to form polynuclear myotubes that visibly twitched when observed under light microscopy. FIG. 10 shows a series of phase contrast microscopy images that show a low magnification (10×) view of the differentiation process at days 1, 3, 4, and 7. A high magnification view of the cells at day 4 clearly shows the presence of multi-nucleated tubular structures. C2C12 myotubes were left as is or treated with 30 ug/mL of the double amino acid gAd variants V207K/I125E and F184H/Y122H for 60 minutes. As controls for this experiment, myotubes were also treated with 30 ug/mL commercial native gAd (BioVision). AICAR (a chemical activator of AMPK) was used as a positive control and an empty vector control lysate (that was processed through the identical chromatography scheme as the gAd variants) was used as the negative control. After treatment, the C2C12 cells were processed into lysate and the amount of both total AMPK and phosphorylated AMPK (pAMPK) was determined by Western blotting with either total or phosphor-speicific AMPK antibodies. FIG. 10 shows that the positive control, AICAR, induced a potent increase in pAMPK, while untreated cells and the vector did not. Commercial native gAd generated a mild increase in pAMPK and the two engineered gAd variants were even more effective. From this experiment we conclude that the gAd variants V207K/I125E and F184H/Y122H have retained biological activity at least equal to or greater than native gAd.

### EXAMPLE 25

### gAd Double Variants Induce AMPK Phosphorylation in Differentiated Human Muscle Cells

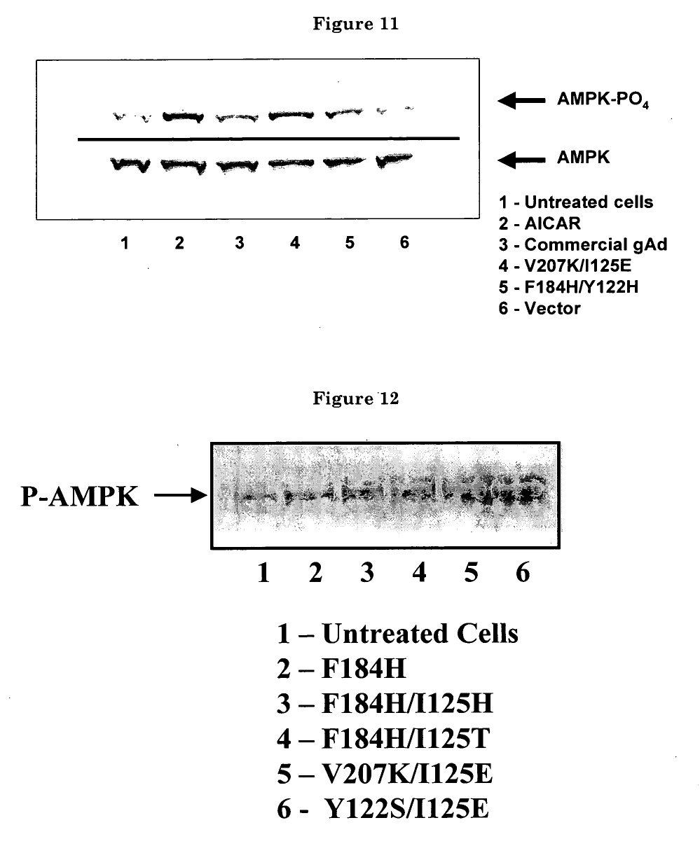

To compliment the above results using murine C2C12 myotubes, we measured the ability of gAd variants to induce pAMPK in differentiated human muscle cells. Pre-screened Human Skeletal Muscle Cells (HSkMC) were obtained from Cell Applications, Inc. and propagated in Skeletal Muscle Cells Growth Medium according to the manufacturer's instructions. To induce differentiation of HSkMC into myotubes, the medium of 90% confluent cell cultures in 6-well plates was replaced by appropriate volume of Skeletal Muscle Differentiation Medium from the same supplier. Differentiation Medium was changed every other day and multinucleated myotubes were observed by the fourth day of differentiation. Differentiation Medium was finally changed 18 hours prior to gAd treatment. On the day of gAd treatment, the cells were washed and incubated in Skeletal Muscle Cells Growth Medium for three hours prior to the addition of adiponectin variants. HSkMC myotubes were left untreated or treated with 50 ug/mL of the gAd variants F184H, F184H/I125H, F184H/I125T, V207K/I125E, and Y122S/I125E for 15 minutes. After the incubation, cells were washed two times with ice-cold PBS, then 200 ml of pre-heated (90° C.) 1×SDS sample buffer supplemented with phosphatase inhibitors was added to each well and the plates were placed on a shaker for two minutes to solubilize the cells and generate a crude cell lysate. This material was harvested and transferred to 1.5 mL eppendorf tubes, heated for an additional 10 minutes at 95° C. and stored overnight at −20° C. On the next day, samples were thawed and passed through a 27-gauge syringe three times followed by centrifugation at 20000 g for 15 min. 20 ml of each sample was loaded on NuPAGE 7% Tris-Acetate. Gel (1.0 mm×10 well) and the gels were run in Tris-Acetate buffer at 150 V constant for 80 min. Upon completion, the gels were incubated in 2× transfer buffer with 0.01% SDS for 20 min followed by transfer to PVDF membranes using 100 V constant for 1 hour. PVDF membranes were incubated with TBS+Tween 20 blocking buffer for 20 min. Anti-Phospho-AMPK antibodies were added in 1:1000 dilution in TBST buffer and membranes were incubated O/N at 40° C. After washes (3 times, 15 min each), membranes were treated with alkaline phosphatase-coupled secondary antibodies for 1 hour at room temperature. Proteins were visualized by using NBT/BCIP alkaline phosphatase substrate. The results of this experiment are presented in FIG. 11; all the variants tested produced an approximately two-fold increase in pAMPK levels relative to the untreated control.

While the foregoing has been described in considerable detail and in terms of preferred embodiments, these are not to be construed as limitations on the disclosure or claims to follow. Modifications and changes that are within the purview of those skilled in the art are intended to fall within the scope of the invention. For example, variants of polypeptides related to adiponectin (e.g., members of the C1q TNF-α superfamily or CTRP family, and their homologs, orthologs or paralogs).can be made using the methods described above.

1-23. (canceled)

24. An adiponectin variant comprising at least one modification relative to a parent adiponectin, wherein the solubility of said variant is improved by at least 3-fold relative to residues 110-244 of human adiponectin (SEQ ID NO: 1).

25. An adiponectin variant of claim 24, wherein said solubility of said variant is improved by at least 5-fold.

26. An adiponectin variant of claim 24, wherein said solubility of said variant is improved by at least 10-fold.

27. An adiponectin variant of claim 24, wherein said solubility of said variant is improved by at least 30-fold.

28. An adiponectin variant of claim 24, wherein said solubility of said variant is improved by at least 60-fold.

29. An adiponectin variant of claim 24, wherein the solubility or soluble expression of said variant is improved by at least 100-fold.

30. An adiponectin variant of claim 24, wherein the solubility or soluble expression of said variant is improved by at least 300-fold.

31. An adiponectin variant of claim 24, wherein the solubility or soluble expression of said variant is improved by at least 1000-fold.

32. An adiponectin variant of claim 24, wherein the expression yield of said variant is improved by at least 2-fold.

33. An adiponectin variant of claim 27, wherein said expression yield of said variant is improved by at least 5-fold.

34. An adiponectin variant of claim 27, wherein said expression yield of said variant is improved by at least 10-fold.

35. An adiponectin variant of claim 27, wherein said expression yield of said variant is improved by at least 50-fold.

36. An adiponectin variant of claim 24, wherein the ability of said variant to induce phosphorylation of AMPK in muscle cells is improved by at least 30% relative to residues 110-244 of (SEQ ID NO: 1).

37. An adiponectin variant of claim 24, wherein the ability of said variant to induce phosphorylation of AMPK in muscle cells is improved by at least 100% relative to residues 110-244 of (SEQ ID NO: 1).

38. An adiponectin variant of claim 24, wherein said parent adiponectin is human adiponectin (SEQ ID NO: 1).

39. An adiponectin variant of claim 24, wherein said parent adiponectin is not human adiponectin(SEQ ID NO: 1).

40. An adiponectin variant of claim 24, wherein said at least one modification is selected from the group consisting of Y109D, Y109E, Y109H, Y109K, Y109N, Y109Q, Y109R, V110D, V110E, V110H, V110K, V110N, V110Q, V110R, V110S, Y111D, Y111E, Y111K, Y111N, Y111Q, Y111R, Y122D, Y122E, Y122H, Y122N, Y122R, Y122S, I125D, I125E, I125H, I125K, I125N, I125Q, I125R, I125S, I125T; M128A, M128D, M128E, M128H, M128K, M128N, M128Q, M128R, M128S, M128T, I135D, I135E, I135H, I135K, I135N, I135Q, I135R, C152A, C152N, C152S, M182A, M182D, M182E, M182K, M182N, M182Q, M182R, M182S, M182T, F184D, F184H, F184K, F184N, F184R, V207D, V207E, V207H, V207K, V207N, V207Q, V207R, V207S, L224D, L224E, L224H, L224K, L224N, L224Q, L224R, L224S, Y225D, Y225E, Y225H, Y225K, Y225N, Y225Q, Y225R, Y225S, D227H, D227K, D227R, D229H, D229K, and, D229R.

41. An adiponectin variant of claim 40, wherein said variant comprises at least two modifications.

42. An adiponectin variant of claim 40, wherein said variant comprises said at least one modification selected from the group consisting of 122H; 122S; 125E; 125H; 125T; 184H; 207E; and 207K.

43. An adiponectin variant of claim 40, wherein said at least one modification is selected from the group consisting of 122H and 122S.

44. An adiponectin variant of claim 40, wherein said at least one modification is selected from the group consisting of 125E, 125H and 125T.

45. An adiponectin variant of claim 40, wherein said at least one modification comprises 184H.

46. An adiponectin variant of claim 40, wherein said at least one modification is selected from the group consisting of 207E and 207K.

47. An adiponectin variant of claim 40, wherein said parent adiponectin consists essentially of residues 110-244 relative to human adiponectin (SEQ ID NO: 1).

48. A composition comprising a polynucleotide encoding an adiponectin variant of claim 24.

49. A composition of claim 48, wherein said polynucleotide encodes an adiponectin variant of claim 47.
